(19) United States
(12) Patent Application Publication
(10) Pub. No.: US 2025/0333688 A1
(43) Pub. Date: Oct. 30, 2025

(54) STRAINS, COMPOSITIONS AND METHODS OF USE

(71) Applicant: L'Oreal S.A., Paris (FR)

(72) Inventors: Charlotte Vedel, Copenhagen (DK); Jens Edward Tesdorpf, Copenhagen (DK); Soren Kjaerulff, Copenhagen (DK); Helena Falholt Elvebakken, Copenhagen (DK)

(73) Assignee: L'Oreal S.A., Paris (FR)

(21) Appl. No.: 18/870,004

(22) PCT Filed: May 26, 2023
(86) PCT No.: PCT/EP2023/064179

§ 371 Date: Nov. 27, 2024

(30) Foreign Application Priority Data
May 31, 2022 (DK) PA 2022 00504
Jan. 31, 2023 (DK) PA 2023 00084

(51) Int. Cl. C12N 1/20; A61K 35/00; A61P 31/10; C12R 1/25

(52) U.S. Cl. CPC C12N 1/205; A61P 31/10; A61K 2035/115; C12R 2001/25

(57) ABSTRACT

The present invention relates to a composition comprising one or more probiotic bacterial strains, wherein the composition is capable of inhibiting, partly or completely proliferation of one or more fungal species.

### TECHNICAL FIELD OF THE INVENTION

The present invention relates to novel lactic acid bacterial strains, which alone or in combination can be used as probiotics. In particular, the present invention relates to new strains, new fungicidal compositions and use of the strains for prevention or treatment of mycoses.

### BACKGROUND OF THE INVENTION

The incidence of fungal infections has increased significantly in the last several decades, this fact is mainly due to the rise in antimicrobial resistance and the limited number of efficient antifungal drugs, which still have many side effects. The use of broad-spectrum antibiotics, denture stomatitis, catheters and parenteral nutrition, the presence of immunosuppression, the disruption of mucosal barriers, and the use of chemotherapy and radiotherapy as well as dysfunctional microbiomes are among the most significant predisposing factors for invasive fungal infections development.

The pathogenicity of fungal species is attributed to certain virulence factors, such as the ability to evade host defences, formation of hyphae, adhesion and biofilm formation (on host tissue or on medical devices), and the production of tissue-damaging hydrolytic enzymes such as proteases, phospholipases, and haemolysins.

Biofilms are biological communities with an extraordinary degree of organization, in which microorganisms form structured, coordinated, and functional communities, embedded in a self-created extracellular matrix. Biofilm production is also associated with a high level of antimicrobial resistance of the associated organisms. The ability of fungal species to form drug-resistant biofilms is an important factor in their contribution to human disease, but it is also recognized that the more general provision of new medical practices (immunosuppressive therapy, invasive surgical procedures, the use of broad-spectrum antibiotics and diseases caused by dysfunctional microbiomes) are highly significant as well. The biofilms are resistant to a range of antifungal agents currently in clinical use, including amphotericin B and fluconazole, and there appear to be multiple resistance mechanisms.

Antifungal drugs are active by either killing the fungal cells, e.g., affecting a substance in the cell wall, causing a leak out of the cell contents and death, or by preventing fungal cells from growing and reproducing. There are many antifungal classes: polyenes, which includes amphotericin, nystatin, and pimaricin; azoles, including fluconazole, itraconazole, ketoconazole, miconazole, voriconazole, posaconazole, and rosaconazole; echinocandins, such as caspofungin and micafungin; and allylamines, including naftifine, terbinafine, morpholine drug, amorolfine, and griseofulvin; and the antimetabolite antifungal drugs, in which 5-fluorocytosine is incorporated. Common for these drugs are the increasing problems with development of resistance, lack of efficiency as well as the toxicity of the compounds.

Amphotericin B and fluconazole are among the antifungal agents most widely used to treat systemic fungal infections. The former has its use limited due to its high degree of toxicity in humans. The latter is frequently prescribed to treat infections, although a great number of fungal species display fluconazole resistance.

Mycoses is in humans and animals, an infection caused by any fungus that invades the tissues, causing superficial, subcutaneous, or systemic disease. Many different types of fungi can cause mycosis, and some types, such as Cryptococcus and Histoplasma, can cause severe, life-threatening infections. Mycosis is a contagious disease caused by a microscopic fungus. The fungus, depending on the type, multiplies in the skin folds, fingernails, hair and mucous membranes, such as the mouth. There are two main types of fungi that cause lesions, and which may be confused with e.g. eczema. Dermatophytes: these fungi attack the keratin found in the skin, fingernails and hair. These actual parasites are transmitted by contact with animals, humans or surfaces. Yeasts (Candida): originate in the mucous membranes and can begin to overproliferate. As a result, lesions develop on the skin, usually around openings. The most common example occurs in the diaper area of infants. Fungal skin infections can happen anywhere on your body. Some of the most common are athlete's foot, jock itch, ringworm and yeast infections.

Superficial fungal infections, also called dermatophytosis, are confined to the skin and are caused by Microsporum, Trichophyton, or Epidermophyton; athlete's foot, for example, is caused by Trichophyton or Epidermophyton. Subcutaneous infections, which extend into tissues under the skin, including adjacent structures such as bone and organs, are rare and often chronic. Candidiasis (caused by Candida) may be a superficial infection (e.g., thrush or vaginitis) or a disseminated infection affecting certain target organs, such as the eyes or kidneys. In sporotrichosis (caused by Sporothrix), painful ulcerations and nodules appear in subcutaneous tissues. In systemic fungal infections, fungi may invade normal hosts or immunosuppressed hosts (causing opportunistic infections).

Cryptococcosis (caused by Cryptococcus) and histoplasmosis (caused by Histoplasmaa) are marked by respiratory distress.

Athlete's foot, also called tinea pedis, is a mycosis of your foot. The fungi grow best in warm, moist places such as shoes, socks, swimming pools, locker rooms, and public showers. They're often found in the summer and in hot, humid climates. It happens more often in people who wear tight shoes, who don't change wet or sweaty socks, and who use public baths and pools.

A type of mycosis called tinea causes jock itch. The infection is also known as tinea cruris. Tinea loves warm, moist areas like your genitals, inner thighs, and buttocks, mycosis happen more often in the summer or in warm, wet climates.

Jock itch is a red, itchy rash that's often ring-shaped. It's only mildly contagious. It can spread from person to person through direct contact or indirectly through objects with the fungus on them.

Ringworm, also called tinea corporis, is not a worm but a fungal skin infection. It is named for its ring-shaped rash with a winding, worm-like edge. Ringworm can spread through direct contact with infected people or animals. You can also pick it up off clothing or furniture. Heat and humidity can help spread the infection.

Mycoses are classified as superficial, cutaneous, subcutaneous, or systemic (deep) infections depending on the type and degree of tissue involvement and the host response to the pathogen.

Cutaneous mycoses (superficial mycoses) are fungal diseases which are confined to the outer layers of the skin, nail and hair (keratinized layers), rarely invading the deeper tissues or viscera. The infection is restricted to the stratum corneum, with little or no tissue reaction. Superficial Mycoses include the following fungal infections and their etiological agent: black piedra (Piedraia hortae), white piedra (Trichosporon beigelii), pityriasis versicolor (Malassezia furfur), and tinea nigra (Phaeoannellomyces werneckii). Pityriasis versicolor is a common superficial mycosis, which is characterized by hypopigmentation or hyperpigmentation of skin of the neck, shoulders, chest, and back. Pityriasis versicolor is due to Malassezia furfur which involves only the superficial keratin layer. Black piedra is a superficial mycosis due to Piedraia hortae which is manifested by a small firm black nodule involving the hair shaft. By comparison, white piedra due to T beigelii is characterized by a soft, friable, beige nodule of the distal ends of hair shafts. Tinea nigra most typically presents as a brown to black silver nitrate-like stain on the palm of the hand or sole of the foot.

Cutaneous Mycoses may be classified as dermatophytoses or dermatomycoses and common infections. Dermatophytoses are caused by the agents of the genera Arthroderma, Lophophyton, Nannizzia, Epidermophyton, Microsporum, Debrayomyces, Kluyveromyces and Trichophyton. Dermatomycoses are cutaneous infections due to other fungi, the most common of which are Candida and Pichia spp. The dermatophytoses are characterized by an anatomic site-specificity according to genera. For example, Epidermophyton floccosum infects only skin and nails, but does not infect hair shafts and follicles. Whereas, Microsporum spp. infect hair and skin, but do not involve nails. Trichophyton spp. may infect hair, skin, and nails.

There are three general types of subcutaneous mycoses: chromoblastomycosis, mycetoma, and sporotrichosis. All appear to be caused by traumatic inoculation of the etiological fungi into the subcutaneous tissue. Chromoblastomycosis is a subcutaneous mycosis characterized by verrucoid lesions of the skin (usually of the lower extremities).

Chromoblastomycosis and mycetoma are caused by only certain fungi. The most common causes of chromoblastomycosis are Fonsecaea pedrosoi, Fonsecaea compacta, Cladosporium carionii, and Phialophora verrucosa.

The causes of mycotic mycetoma are more diverse but can be classified as eumycotic and actinomycotic mycetoma. Common fungi of eumycotic mycetoma is Pseudallescheria boydii and the most common cause of actinomycotic mycetoma is Nocardia brasiliensis. These fungi may produce a range of infections from superficial to subcutaneous to deep (visceral) infection characterized by the presence of dematiaceous hyphal and/or yeast-like cells in tissue. Such deep infections due to dematiaceous fungi are termed phaeohyphomycosis.

Sporotrichosis is the third general class of subcutaneous mycoses. This infection is due to Sporothrix schenckii and involves the subcutaneous tissue at the point of traumatic inoculation. The infection usually spreads along cutaneous lymphatic channels of the extremity involved.

Deep mycoses are caused by primary pathogenic and opportunistic fungal pathogens. The primary pathogenic fungi are able to establish infection in a normal host; whereas, opportunistic pathogens require a compromised host in order to establish infection (e.g. cancer, organ transplantation, surgery, and AIDS). The primary deep pathogens usually gain access to the host via the respiratory tract. Opportunistic fungi causing deep mycosis invade via the respiratory tract, alimentary tract, or intravascular devices.

The primary systemic fungal pathogens include Coccidioides immitis, Histoplasma capsulatum, Blastomyces dermatitidis, and Paracoccidioides brasiliensis. The opportunistic fungal pathogens include Cryptococcus neoformans, Candida spp., Aspergillus spp., Penicillium marneffei, the Zygomycetes, Trichosporon beigelii, and Fusarium spp.

Topical antifungal drugs, usually available as creams, liquids, or sprays, are often ineffective for superficial infections. The antifungal griseofulvin has met with some success in the treatment of superficial mycoses, and amphotericin B and flucytosine have been used in treating subcutaneous and systemic mycoses. But more and more mycoses are difficult to treat.

Athlete's foot is an infection caused by a type of fungus known as a dermatophyte. Able to infect only the top layer of dead keratin, dermatophytes affect the skin, hair shafts, and nails. Dermatophytes are classified into three genera: Trichophyton, Microsporum, and Epidermophyton. T. rubrum is the dermatophyte most commonly associated with athlete's foot. Although other dermatophytes can also cause the condition, they are less frequently isolated from humans. Fungal spores from T. rubrum can live in human scales for 12 months and are therefore easily transmitted from person to person in locker rooms and public showers.

Antifungal resistance is both complex and multifaceted. It can be inducible in response to a compound or be an irreversible genetic change resulting from prolonged exposure. In detail, these include alterations or even an overexpression of target molecules, active extrusion through efflux pumps, limited diffusion, tolerance, and cell density, which are all characterized mechanisms utilized by fungi to combat the effects of antifungal treatments. Planktonic cells generally rely on irreversible genetic changes to maintain a resistant phenotype, whereas biofilm cells are able to persist due to their physical presence and the density of the population, which provides an almost inducible resistant phenotype irrespective of defined genetic alterations.

Fungal species belong to the normal microbiota of the oral cavity, skin, gastrointestinal and vaginal tracts, and are responsible for several clinical manifestations, from mucocutaneous overgrowth to bloodstream infections. Year after year, the pathological circumstances caused by the fungal microbiota are more recurrent and problematic to treat, especially when patients reveal any level of immunosuppression, have received antibiotics or has a dysfunctional microbiome e.g. with decreased microbial diversity.

Lactic acid bacteria are a part of the microbial flora of the human gut, mouth, and vagina. Lactic acid bacteria play an important role in protecting the human body from infection via production of acids and acidification of the e.g. vagina, by production of other antimicrobial products, such as hydrogen peroxide H₂O₂, antimicrobial peptides or biosurfactants. Many topical, vaginal, oral and systemic medications may kill lactic acid bacteria. Hence, treatment of infections with antibiotics may place the body at increased risk for repeated acquisition of the infection or new infections caused by resistant microorganism e.g. fungi.

For some decades, antifungal agents have been successfully used to prevent mucosal as well as invasive fungal infections. However, due to the drug side effects (nausea, vomiting, and diarrhea) and the emergence of resistant strains, antifungal prophylaxis has not been totally successful. The limited effect and the gradual emergence of resistance to antifungal drugs are a concern, thus alternative therapies are urgently warranted.

Use of probiotic microorganisms against mycoses may be an attractive alternative therapeutic to treat or prevent mycoses in view of the limitation of the currently available antimicrobial compounds.

Lactic acid bacteria for intravaginal or oral use have been available for over 50 years in the form of probiotic preparations available in health food stores or as dairy products. Typically the traditional probiotics are used to maintain a healthy gut flora and not known for any targeted or specific antimicrobial mechanisms.

The present invention provides new probiotic strains and compositions which are targeted to at least one pathogenic fungi, preferably a fungi causing mycosis, in particular the composition may provide new probiotic strains and compositions inhibiting proliferation of dermatophytes and showing general antifungal activity causing growth inhibition, inhibition of spore germination, inhibition of hyphae formation, inhibition of biofilm attachment or formation or the ability to prevent fungi their potency to secrete virulence factors as phospholipase, proteinase and haemolytic factor, and/or prevent and/or treat mycosis in a human or animal.

### SUMMARY OF THE INVENTION

Thus, an object of the present invention relates to new lactic acid bacteria as well as compositions, topical compositions, vaginal compositions, eye compositions, ear compositions rectal composition or oral compositions comprising said new lactic acid bacteria, and their ability to inhibiting proliferation of dermatophytes, inhibit growth, inhibition of spore germination, inhibition of hyphae formation, inhibit fungal proliferation, and/or prevent or treat mycoses.

In particular, it is an object of the present invention to provide a new lactic acid bacterial strain and compositions comprising this new lactic acid bacterial strain that solves the above-mentioned problems of the prior art, in particular new lactic acid bacteria with antifungal activity, and thereby the ability to prevent mycoses, and preferably without the mentioned side effects.

Thus, one aspect of the invention relates to a composition comprising Lactiplantibacillus plantarum, LB990R, deposited under the assession number DSM 34494 by Lactobio A/S on Jan. 10, 2023; Lactiplantibacillus plantarum, LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum, LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; or a combination hereof.

Yet another aspect of the present invention relates to a composition comprising one or more probiotic bacterial strains, wherein the composition is capable of inhibiting, partly or completely proliferation of one or more fungal species, in particular one or more dermatophyte.

A further aspect of the present invention relates to an isolated probiotic bacterial strain selected from:
Lactiplantibacillus plantarum, LB990R, deposited under the assession number DSM 34494; and/or
Lactiplantibacillus plantarum, LB681R, deposited under the assession number DSM 34250; and/or
Lacticaseibacillus paracasei LB857R, deposited under the assession number DSM 34493; and/or
Lactiplantibacillus plantarum LB760R, deposited under the assession number DSM 34492; and/or
Lacticaseibacillus paracasei subsp. paracasei LB555R deposited under the assession number DSM 34249; and/or

Yet an aspect of the present invention relates to an isolated probiotic bacterial strain selected from:
Lactiplantibacillus plantarum LB990R, deposited under the assession number DSM 34494 by Lactobio A/S on Jan. 10, 2023;
Lactiplantibacillus plantarum LB681R, deposited under the assession number DSM 34250 by Lactobio A/S on May 5, 2022;
Lacticaseibacillus paracasei LB857R, deposited under the assession number DSM 34493 by Lactobio A/S on Jan. 10, 2023;
Lactiplantibacillus plantarum LB760R, deposited under the assession number DSM 34492 by Lactobio A/S on Jan. 10, 2023;
Lacticaseibacillus paracasei subsp. paracasei LB555R, deposited under the assession number DSM 34249 by Lactobio A/S on May 5, 2022.

capable of inhibiting, partly or completely the proliferation of one or more dermatophyte.

Another aspect of the present invention relates to a composition comprising Lacticaseibacillus paracasei subsp. paracasei LB555R deposited under the assession number DSM 34249; Lactiplantibacillus plantarum LB681R, deposited under the assession number DSM 34250; Lactiplantibacillus plantarum LB760R, deposited under the assession number DSM 34492; Lactiplantibacillus plantarum LB990R, deposited under the assession number DSM 34494; Lacticaseibacillus paracasei LB857R, deposited under the assession number DSM 34493; or a combination hereof, for use in:
the treatment or prevention of mycoses, preferably the treatment of and/or prevention of mycoses in a human or in an animal;
the treatment of dermatophytosis, preferably the treatment of and/or prevention of dermatophytosis in a human or in an animal; and/or
the prevention, inhibition, and/or treatment of fungal growth on crops, seeds, food or feed.

Still another aspect of the present invention relates to a composition comprising one or more bacterial strains selected from one or more lactic acid bacteria for use in the prevention and/or treatment of a microbiota dysbiosis caused by fungi.

Still another aspect of the present invention relates to a composition comprising one or more bacterial strains selected from one or more lactic acid bacteria for use in the prevention and/or treatment of mycosis in a human or in an animal.

Still another aspect of the present invention relates to a composition comprising one or more bacterial strains selected from Lactiplantibacillus plantarum LB990R, deposited under the assession number DSM 34494; Lactiplantibacillus plantarum LB681R, deposited under the assession number DSM 34250; Lacticaseibacillus paracasei LB857R, deposited under the assession number DSM 34493; Lactiplantibacillus plantarum LB760R, deposited under the assession number DSM 34492; Lacticaseibacillus paracasei subsp. paracasei LB555R deposited under the assession number DSM 34249 or a combination hereof for use in the prevention and/or treatment of mycoses in a human or in an animal.

Still another aspect of the present invention relates to a composition comprising one or more bacterial strains selected from one or more lactic acid bacteria for use in the prevention and/or treatment of fungal infections or contaminations in corps, foods, or animal feed.

A further aspect of the present invention relates to an antifungal composition comprising one or more probiotic bacterial strains for the inhibition of spore germination of a fungus and/or inhibition of hyphae formation of a fungus.

Still another aspect of the present invention relates to use of a composition according to the present invention comprising one or more bacterial strains selected Lactiplantibacillus plantarum LB990R, deposited under the assession number DSM 34494; Lactiplantibacillus plantarum LB681R, deposited under the assession number DSM 34250; Lacticaseibacillus paracasei LB857R, deposited under the assession number DSM 34493; Lactiplantibacillus plantarum LB760R, deposited under the assession number DSM 34492; Lacticaseibacillus paracasei subsp. paracasei LB555R deposited under the assession number DSM 34249 or a combination hereof) in the prevention, inhibition, or treatment of fungal growth on crops, seeds, food or feed.

A further aspect of the present invention relates to a composition comprising one or more bacterial strains selected from one or more lactic acid bacteria for use in the prevention and/or treatment of mycosis in a human or in an animal.

A further aspect of the present invention relates to a method for reducing and/or inhibiting spore germination of a fungus and/or hyphae formation of a fungus, the method comprises addition of a composition according to the present invention, to a surface infected with fungi or prevent the surface to be infected with fungi.

An even further aspect of the present invention relates to a method for preventing mycosis in an environment, wherein the method comprising the steps of: administering to the environment an effective amount of a lactic acid bacteria with antifungal activity, wherein the environment is a home, workplace, laboratory, industrial environment, hospital environment, aquatic environment, medical device, dental device, plants, corps, epithelial cells, mucous membranes, an animal or a human body.

Still another aspect of the present invention relates to a method for preventing fungal growth in an environment comprising the steps of: administering to the environment an effective amount of a lactic acid bacteria with antifungal activity, wherein the environment is selected from a home, a workplace, a laboratory, an industrial environment, an aquatic environment, a medical device, or a dental device.

A further aspect of the present invention relates to a food or feed ingredient or preservative, and a personal hygiene product, comprising the compositions according to the invention.

### DETAILED DESCRIPTION OF THE INVENTION

Accordingly, the inventors of the present invention found particular lactic acid bacteria having antifungal activity targeted towards pathogenic fungi.

It is believed that the lactic acid bacteria of the present invention are able to inhibit or prevent mycoses.

A preferred embodiment of the present invention relates to a composition comprising one or more probiotic bacterial strains, wherein the composition is capable of inhibiting, partly or completely proliferation of one or more fungal species.

A further preferred embodiment of the present invention relates to a composition comprising one or more probiotic bacterial strains, wherein the composition may be capable of inhibiting, partly or completely the proliferation of one or more dermatophyte.

The one or more dermatophyte may preferably be selected from Arthroderma, Lophophyton, Nannizzia, Epidermophyton, Microsporum, Debrayomyces, Kluyveromyces, Pichia, Trichophyton, or a combination hereof.

Preferably, the probiotic bacterial strains may be selected from at least one lactic acid bacterial strain.

The one or more probiotic bacterial strains may preferably be one or more isolated probiotic bacterial strains.

The at least one lactic acid bacterial strain may preferably be selected from one or more Lacticaseibacillus strains; and/or one or more bacteria strains selected from one or more Lactiplantibacillus strains; or a combination hereof.

Preferably, the one or more probiotic bacterial strains may be selected from a Lacticaseibacillus paracasei and/or a Lactiplantibacillus plantarum.

The one or more lactic acid bacterial strains may be selected from Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; or a combination hereof.

In an embodiment of the present invention the composition may be a composition comprising one or more lactic acid bacteria according to the present invention together with an acceptable carrier and/or diluent.

Preferably, the composition comprising 10³ to 10¹³ colony forming units of lactic acid bacteria according to the present invention per gram. More specifically a composition comprising 10⁴ to 10¹² colony forming units of lactic acid bacteria per gram. More specifically a composition comprising 10⁵ to 10¹¹ colony forming units of lactic acid bacteria per gram.

The composition may be in the form of a suspension, spray, gel, cream, lotion, powder, capsule, oil, solution for lavages, ovules, a vaginal insert, a suppository, lozenge, tablets, microencapsulated product or in form of a food supplement or a food product.

In an embodiment of the present invention the composition may be a pharmaceutical composition comprising one or more lactic acid bacteria according to the present invention together with a pharmaceutically acceptable carrier and/or diluent.

Preferably, the pharmaceutical composition comprising 10³ to 10¹³ colony forming units of lactic acid bacteria according to the present invention per gram. More specifically a pharmaceutical composition comprising 10⁶ to 10¹² colony forming units of lactic acid bacteria per gram. More specifically a pharmaceutical composition comprising 10⁷ to 10¹¹ colony forming units of lactic acid bacteria per gram.

The pharmaceutical composition may be in the form of a suspension, a spray, a gel, a cream, a lotion, a powder, a capsule, an oil, a solution for lavages, an ovules, a vaginal insert, a suppository, a lozenge, a tablet, a bandage, a band aid, a microencapsulated product or in form of a food supplement or a food product.

In an embodiment of the present invention the composition may be a dermatological composition comprising one or more lactic acid bacteria according to the present invention together with a dermatological acceptable carrier and/or diluent.

Preferably, the dermatological composition comprising 10³ to 10¹³ colony forming units of lactic acid bacteria according to the present invention per gram. More specifically a dermatological composition comprising 10⁶ to 10¹² colony forming units of lactic acid bacteria per gram. More specifically a dermatological composition comprising 10⁷ to 10¹¹ colony forming units of lactic acid bacteria per gram.

The dermatological composition may be in the form of a suspension, a spray, a gel, a cream, a powder, or a lotion.

The composition may be provided with a therapeutic effective amount of the lactic acid bacteria, in particular a therapeutic effective amount of the one or more bacteria selected from:
Lactiplantibacillus plantarum LB990R, deposited under the assession number DSM 34494 by Lactobio A/S on Jan. 10, 2023; or
Lactiplantibacillus plantarum, LB681R, deposited under the assession number DSM 34250 by Lactobio A/S on May 5, 2022; or
Lacticaseibacillus paracasei LB857R, deposited under the assession number DSM 34493 by Lactobio A/S on Jan. 10, 2023; or
Lactiplantibacillus plantarum LB760R, deposited under the assession number DSM 34492 by Lactobio A/S on Jan. 10, 2023; or
Lacticaseibacillus paracasei subsp. paracasei LB555R, deposited under the assession number DSM 34249 by Lactobio A/S on May 5, 2022; or
a combination hereof.

The composition may be provided with a therapeutic effective amount of the lactic acid bacteria selected from Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; or a combination hereof, further in combination with at least one further lactic acid bacteria.

In the context of the present invention the terms “therapeutic effective amount” or “effective amount” may refer to an amount of the compounds in a composition or preparation which, when administered as part of a desired dosage regimen (to a human or an animal, preferably a human) alleviates a symptom, ameliorates a condition, or slows the onset of disease conditions according to clinically acceptable standards for the disorder or condition to be treated or the cosmetic purpose, e.g., at a reasonable benefit risk ratio applicable to any medical treatment.

An embodiment of the present invention relates to a composition comprising Lacticaseibacillus paracasei subsp. paracasei LB555R deposited under the assession number DSM 34249; Lactiplantibacillus plantarum LB681R, deposited under the assession number DSM 34250; Lactiplantibacillus plantarum LB760R, deposited under the assession number DSM 34492; Lactiplantibacillus plantarum LB990R, deposited under the assession number DSM 34494; Lacticaseibacillus paracasei LB857R, deposited under the assession number DSM 34493; or a combination hereof, for use in:
the treatment or prevention of mycoses, preferably the treatment of and/or prevention of mycoses in a human or in an animal;
the treatment of dermatophytosis, preferably the treatment of and/or prevention of dermatophytosis in a human or in an animal; and/or
the prevention, inhibition, and/or treatment of fungal growth on crops, seeds, food or feed.

For the practical use, the microorganisms of the invention may be formulated in suitable administration forms such as a suspension, a spray, a gel, a cream, a lotion, a powder, a capsule, an oil, a solution for lavages, an ovules, a vaginal insert, a suppository, a lozenge, a tablet, a microencapsulated product or in form of a food supplement or a food product and the like. Unit doses may comprise from 10 to 10¹³ cells of each single strain, the preferred dosage being above 10⁴ cells per unit dose. More preferable dosage is above 10⁵ cells per unit dose.

The bacterial cultures may be stabilized in freeze dried, lyophilized or microencapsulated forms and may be prepared according to conventional methods.

For the preparation of aqueous formulations for lavages and irrigations a two-phase system can be used. E.g. small bottles with reservoirs containing the lyophilized microorganisms, to be dissolved before use in a suitable liquid carrier contained in the bottles.

For the preparation of creams and gels, the lactic acid bacteria may be stabilized in the formulation. Preferable in formulations with low water activity or in a microencapsulated form.

For the preparation of capsule or tablet formulations for oral consumption the lactic acid bacteria may be stabilized using capsule and tablet technology known in the art for lactic acid bacteria stabilization E.g. the technology used for probiotics as food supplements.

In an embodiment of the present invention the one or more lactic acid bacteria strain according to the present invention may be provided in a composition suitable for preventing and/or treating vaginal mycosis.

In yet an embodiment of the present invention the one or more lactic acid bacteria strain according to the present invention may be provided in a composition for preventing and/or treating urinary-tract infections.

In a further embodiment of the present invention the one or more lactic acid bacteria strain according to the present invention may be provided in a composition for preventing and/or treating topical, skin, nail, eye, ear, oral, intestinal, gut or mucosal infections.

In still a further embodiment of the present invention the one or more lactic acid bacteria strain according to the present invention may be provided in a composition for preventing and/or treating oral mycoses.

A further embodiment of the present invention the composition and/or the one or more lactic acid bacteria according to the present invention may be used in combination with a medical device.

Yet an embodiment of the present invention relates to a method for treating or preventing microbial imbalances in mucous membranes in a human or in an animal comprising administration of an effective amount of lactic acid bacteria according to the invention.

In the context of the present invention the term “prevent” may be art-recognized, and when used in relation to a condition, such as a local recurrence, may be well understood in the art, and includes administration of a composition which reduces the frequency of, or delays the onset of, symptoms of a medical condition in a subject relative to a subject which does not receive the composition. Thus, prevention of infection includes, for example, reducing the number of detectable pathogenic microorganisms in a population of patients receiving a prophylactic treatment relative to an untreated control population, and/or delaying the appearance of detectable lesions in a treated population versus an untreated control population, e.g., by a statistically and/or clinically significant amount.

As used herein, the terms “treating” or ‘treatment” includes reversing, reducing, or arresting the symptoms, clinical signs, and underlying pathology of a condition in a manner to improve or stabilize a subject's condition.

As used herein, and as well-understood in the art, “treatment” may be an approach for obtaining beneficial or desired results, including clinical results. For purposes of this subject matter, beneficial or desired clinical results may include, but are not limited to, alleviation or amelioration of one or more symptoms, diminishment of extent of disease, stabilized (i.e., not worsening) state of disease, prevention of disease, delay or slowing of disease progression, and/or amelioration or palliation of the disease state.

The decrease can be at least a 10% decrease in severity of complications or symptoms relative to the state before the treatment, such as at least 20% decrease, e.g. at least 30% decrease, such as at least 40% decrease, e.g. at least 50% decrease, such as at least 60% decrease, e.g. at least 70% decrease, such as at least 80% decrease, e.g. at least 90% decrease, such as at least 95% decrease, e.g. at least 98% decrease, such as at least 99% decrease, e.g. 100% decrease in severity of complications or symptoms.

Mycoses may be fungal infection of a human and an animal and may be caused by invasion of human or animal tissue by one or more fungal species. Mucosis may be superficial mycosis, subcutaneous mycosis, or systemic mucosis.

The fungal species (causing mycosis) may be any fungal species causing mycosis. In particular the fungal species may be selected from the group consisting of Cryptococcus and Histoplasma, (which may cause severe, life-threatening infections), Candida, Microsporum, Trichophyton, or Epidermophyton, Sporothrix (mainly superficial mycosis), Piedraia hortae, Trichosporon beigelii, pityriasis versicolor, Phaeoannellomyces werneckii, Arthroderma, Lophophyton, Nannizzia, Epidermophyton, Microsporum, Debrayomyces, Kluyveromyces, Fonsecaea pedrosoi, Fonsecaea compacta, Cladosporium carionii, Phialophora verrucose, Pseudallescheria boydii, Nocardia brasiliensis, Sporothrix schenckii, Coccidioides immitis, Histoplasma capsulatum, Blastomyces dermatitidis, Paracoccidioides brasiliensis, Cryptococcus neoformans, Candida spp., Aspergillus spp., Penicillium marneffei, Zygomycetes, Trichosporon beigelii, and Fusarium spp. or a combination hereof.

The composition according to the present invention may inhibit, prevent, or treat mycosis anywhere on the human or animal body, such as on the skin, in folds of the skin, on nails, on fingernails, in the eye, in the ear, in the hair, on the feet, on the hands, and in the intestine, gut or mucous membranes, such as the mouth.

The composition according to the present invention may be suitable for inhibit, prevent or treat tinea infections like tinea barbae, tinea capitis, tinea corporis, tinea cruris, tinea faciei, tinea incognito, tinea manuum, tinea pedis, tinea unguium.

The composition according to the present invention may be suitable for inhibit, prevent or treat tinea infections like:
tinea pedis, also called Athlete's foot, which is a mycosis of the foot,
tinea cruris, also known as jock itch which is a mycosis of the groin region,
tinea corporis, also called ringworm, which is a mycosis of the skin and may appear as a red, itchy, scaly, circular rash and hair loss may occur in the affected area.

In an embodiment of the present invention the fungal species may not include Malassezia spp.

In a further embodiment of the present invention the fungal species may not include Candida spp.

“Cutaneous mycoses” (superficial mycoses) are fungal diseases which may be confined to the outer layers of the skin, nail and hair (keratinized layers).

In an embodiment of the present invention the one or more probiotic bacterial strains may be one or more lactic acid bacterial strains.

The term “lactic acid bacteria” includes species from the families Lactobacilliae, Aerococcaceae, Bifidobacteriaceae, Carnobacteriaceae, Enterococcaceae, Leuconostocaceae and Streptococcaceae. These are considered non-pathogenic and are used as probiotic bacteria in general to improve gastrointestinal flora and in the treatment of gastrointestinal symptoms. Lactic acid bacteria according to the present invention may preferably be selected from a lactic acid bacterial strain inhibiting, preventing or reducing mycosis fungal growth. In particular, mycoses in mammals.

The inventors of the present invention surprisingly found certain strains of lactic acid bacteria showed to be able to inhibit, preventing or reducing fungal growth.

Preferably, the present invention relates to an antifungal composition comprising one or more probiotic bacterial strains for the inhibition of spore germination of a fungus and/or inhibition of hyphae formation of a fungus.

Preferably, the fungus may be a dermatophyte.

In an embodiment of the present invention the one or more probiotic bacterial strains is selected from one or more Lactiplantibacillus plantarum, one or more Lacticaseibacillus paracasei, or a combination hereof.

Preferably, the one or more probiotic bacterial strains may be one or more isolated probiotic bacterial strains.

In an embodiment of the present invention the one or more probiotic bacterial strains may be selected from one or more of:
Lactiplantibacillus plantarum LB990R, deposited under the assession number DSM 34494; and/or
Lactiplantibacillus plantarum LB681R, deposited under the assession number DSM 34250;and/or
Lacticaseibacillus paracasei LB857R, deposited under the assession number DSM 34493; and/or
Lactiplantibacillus plantarum LB760R, deposited under the assession number DSM 34492; and/or
Lacticaseibacillus paracasei subsp. paracasei LB555R deposited under the assession number DSM 34249.
A combination hereof.

A preferred embodiment of the present invention may relate to a composition comprising Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022;

Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022;
or a combination hereof.

The composition according to the present invention may comprise viable or non-viable cells of lactic acid bacteria according to the present invention.

The composition according to the present invention may comprise metabolites produced by the lactic acid bacteria of the present invention and/or actives obtained from the lactic acid bacteria of the present invention.

In an embodiment of the present invention the composition comprises a concentration of short chain fatty acids (acetic acid) less than 45 mM determined as the concentration of acetic acid, such as less than 44 mM, e.g. less than 43 mM, such as less than 42 mM, e.g. less than 41 mM, such as less than 40 mM, e.g. less than 39 mM, such as in the range of 25-45 mM, e.g. in the range of 28-44 mM, such as in the range of 30-43 mM, e.g. in the range of 32-42 mM, such as in the range of 34-41 mM, e.g. in the range of 36-40 mM, such as in the range of 38-39 mM. Preferably, the composition may be a postbiotic.

The composition according to the present invention may comprise one or more viable probiotic bacterial strains, or the composition comprises non-viable probiotic bacterial strains or the composition comprises metabolites, lysate, e.g. a ferment-lysate obtained from the one or more probiotic bacterial strains, or the composition comprises a fraction of the one or more probiotic bacterial strains, or a combination hereof.

In an embodiment of the present invention the one or more Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; or a combination hereof may be provided as viable cells, as non-viable cells, as a lysate, as a fraction, as a ferment, as a ferment-lysate, as metabolites, as an extract, as a derivative, as analogues, or as a mutant of one of the strains according to the present invention. Preferably, the composition comprises viable cells or a combination of non-viable cells and metabolites form said cells.

The one or more probiotic bacterial strains may be one or more isolated probiotic bacterial strains.

The term isolated may relate to a probiotic bacterial strain that it is not a wild type ferment but isolated from a natural source and fermented as a single microorganism. Preferably, the isolated probiotic bacterial strain according to the present invention may be produced by fermentation as an individual bacterial strain.

Preferably, the one or more lactic acid bacteria (such as Lactiplantibacillus plantarum LB990R, deposited under the assession number DSM 34494 by Lactobio A/S on Jan. 10, 2023); and/or the one or more lactic acid bacteria (such as Lactiplantibacillus plantarum LB681R, deposited under the assession number DSM 34250 by Lactobio A/S on May 5, 2022); and/or the one or more lactic acid bacteria (such as Lacticaseibacillus paracasei LB857R, deposited under the assession number DSM 34493 by Lactobio A/S on Jan. 10, 2023); and/or the one or more lactic acid bacteria (such as Lactiplantibacillus plantarum LB760R, deposited under the assession number DSM 34492 by Lactobio A/S on Jan. 10, 2023); the one or more lactic acid bacteria (such as Lacticaseibacillus paracasei subsp. paracasei LB555R, deposited under the assession number DSM 34249 by Lactobio A/S on May 5, 2022); the one or more lactic acid bacteria may be provided as viable cells, as non-viable cells, or as a lysate or ferment-lysate or postbiotic.

The composition according to the present invention may be formulated for oral use, topical use, anal use or genital use.

An embodiment of the present invention relates to a composition comprising viable/live probiotic strains according to the present invention for use in treating; alleviating, suppressing; prophylaxis; and/or preventing growth of a pathogenic fungi.

More preferably the present invention may provide a composition as defined herein for use in the treatment, alleviating, suppressing; prophylaxis of one or more pathogenic bacterial infection in a human or an animal.

Yet an embodiment of the present invention relates to a composition comprising Lactiplantibacillus plantarum LB990R, deposited under the assession number DSM 34494; Lactiplantibacillus plantarum LB681R, deposited under the assession number DSM 34250; Lacticaseibacillus paracasei LB857R, deposited under the assession number DSM 34493; Lactiplantibacillus plantarum LB760R, deposited under the assession number DSM 34492; Lacticaseibacillus paracasei subsp. paracasei LB555R deposited under the assession number DSM 34249; or a combination hereof for use against mycoses.

Yet an embodiment of the present invention relates to a composition comprising Lactiplantibacillus plantarum LB990R, deposited under the assession number DSM 34494; Lactiplantibacillus plantarum LB681R, deposited under the assession number DSM 34250; Lacticaseibacillus paracasei LB857R, deposited under the assession number DSM 34493; Lactiplantibacillus plantarum LB760R, deposited under the assession number DSM 34492; Lacticaseibacillus paracasei subsp. paracasei LB555R deposited under the assession number DSM 34249 or a combination hereof for use against cutaneous mycoses caused by dermatophytes (dermatophytosis).

In a further embodiment of the present invention relates to a composition comprising one or more bacterial strains selected from Lactiplantibacillus plantarum LB990R, deposited under the assession number DSM 34494; Lactiplantibacillus plantarum LB681R, deposited under the assession number DSM 34250; Lacticaseibacillus paracasei LB857R, deposited under the assession number DSM 34493; Lactiplantibacillus plantarum LB760R, deposited under the assession number DSM 34492; Lacticaseibacillus paracasei subsp. paracasei LB555R deposited under the assession number DSM 34249; or a combination hereof for use in the prevention and/or treatment of mycoses in a human or in an animal.

In particular the composition according to the present invention comprising one or more bacterial strains selected from Lactiplantibacillus plantarum LB990R, deposited under the assession number DSM 34494; Lactiplantibacillus plantarum LB681R, deposited under the assession number DSM 34250; Lacticaseibacillus paracasei LB857R, deposited under the assession number DSM 34493; Lactiplantibacillus plantarum LB760R, deposited under the assession number DSM 34492; Lacticaseibacillus paracasei subsp. paracasei LB555R deposited under the assession number DSM 34249 or a combination hereof may be suitable for use in treatment of dermatophytosis on epithelial cells, mucous membranes, or on (or in) the human or animal body.

In particular the composition according to the present invention comprising one or more bacterial strains selected from Lactiplantibacillus plantarum, LB990R, deposited under the assession number DSM 34494; Lactiplantibacillus plantarum, LB681R, deposited under the assession number DSM 34250; Lacticaseibacillus paracasei LB857R, deposited under the assession number DSM 34493; Lactiplantibacillus plantarum, LB760R, deposited under the assession number DSM 34492; Lacticaseibacillus paracasei subsp. paracasei LB555R deposited under the assession number DSM 34249; or a combination hereof may be suitable for use in treatment of mycoses on epithelial cells, mucous membranes, or on (or in) the human or animal body.

In an embodiment of the present invention the animal may include, but are not limited to, primates, farm animals, sport animals, rodents and pets. More specifically the animals may include mice, rats, hamsters, and guinea pigs; rabbits; dogs; cats; sheep; pigs; piglets; sows; poultry; turkeys; broilers; minks; goats; cattle; horses; and non-human primates such as apes and monkeys.

More preferably the present invention may provide a composition as defined herein for use in preventing growth of a pathogenic micro-organism.

A “decrease” in growth may be “statistically significant” as compared to the growth period in the absence of the bacterial strains of the present invention, and may include a 10 percent, 20 percent, 30 percent, 40 percent, 50 percent, 60 percent, 70 percent, 80 percent, 85 percent, 90 percent, 95 percent, 99 percent or 100 percent decrease.

In an embodiment of the present invention the growth inhibition may be determined as a decrease in growth of at least 25 percent. Preferably the growth inhibition is determined as a decrease in growth of at least 50 percent, more preferably the growth inhibition is determined as a decrease in growth of at least 80 percent more preferably the growth inhibition is determined as a decrease in growth of at least 90 percent.

“Probiotics” may be defined as live microorganisms that, when administrated or consumed in adequate quantities, confer health benefits on the host.

In a preferred embodiment the lactic acid bacteria of the invention may be a live, viable strain.

The non-viable cells according to the present invention may be provided as a lysate, as a fraction, as a ferment, as a ferment-lysate, as metabolites, as an active, as an extract, as a derivative, as analogues, or as a mutant of one of the strains according to the present invention.

“Lysates”, “ferment-lysate”, “derivatives”, “analogues”, “fractions”, “postbiotic” or “extracts” may be obtained from dead or killed lactic acid bacteria. These lysates, fractions, derivative, analogues, and extracts preferably have the properties of being able to bind or co-aggregate with pathogenic fungi thereby preventing growth and/or attachment and/or hyphae formation and/or biofilm formation of a fungi, where “lysate” as well as the term “extract” and “postbiotic” refers in particular to a solution or suspension in an aqueous medium of the cells of the microorganism and/or metabolites according to the invention and comprises, for example, macromolecules such as DNA, RNA, proteins, peptides, lipids, carbohydrates, acids, metabolites produced by the lactic acid bacteria etc. as well as cell detritus. The lysate preferably includes the cell wall or cell wall constituents including binding receptors. In a preferred embodiment the cells are paraprobiotics not lysed but intact dead cells. Methods of producing lysates are sufficiently well known to those skilled in the art and includes, for example, the use of a French press or enzymatic lysis, a ball mill with glass beads or iron beads. Cells can be broken open by enzymatic, physical or chemical methods. Examples of enzymatic cell lysis may include individual enzymes as well as enzyme cocktails, for example, proteases, proteinase K, lipases, glycosidases; chemical lysis may be induced by ionophores, detergents such as SDS, acids or bases; physical methods may also be implemented by using high pressures such as the French press, osmolarities, temperatures or alternating between heat and cold. Furthermore chemical, physical and enzymatic methods may of course be combined to in-activate the lactic acid bacteria. “Extract” can be any of these cellular components, metabolites, postbiotics, ferment, or cell fractions.

A preferred embodiment of the present invention relates to an isolated probiotic bacterial strain selected from:
Lactiplantibacillus plantarum LB990R, deposited under the assession number DSM 34494 by Lactobio A/S on Jan. 10, 2023; and/or
Lactiplantibacillus plantarum LB681R, deposited under the assession number DSM 34250 by Lactobio A/S on May 5, 2022; and/or
Lacticaseibacillus paracasei LB857R, deposited under the assession number DSM 34493 by Lactobio A/S on Jan. 10, 2023; and/or
Lactiplantibacillus plantarum LB760R, deposited under the assession number DSM 34492 by Lactobio A/S on Jan. 10, 2023; and/or
Lacticaseibacillus paracasei subsp. paracasei LB555R, deposited under the assession number DSM 34249 by Lactobio A/S on May 5, 2022.

A further preferred embodiment of the present invention relates to an isolated probiotic bacterial strain selected from:
Lactiplantibacillus plantarum LB990R, deposited under the assession number DSM 34494 by Lactobio A/S on Jan. 10, 2023;
Lactiplantibacillus plantarum LB681R, deposited under the assession number DSM 34250 by Lactobio A/S on May 5, 2022;
Lacticaseibacillus paracasei LB857R, deposited under the assession number DSM 34493 by Lactobio A/S on Jan. 10, 2023;
Lactiplantibacillus plantarum LB760R, deposited under the assession number DSM 34492 by Lactobio A/S on Jan. 10, 2023;
Lacticaseibacillus paracasei subsp. paracasei LB555R, deposited under the assession number DSM 34249 by Lactobio A/S on May 5, 2022;
   capable of inhibiting, partly or completely the proliferation of one or more dermatophyte.

A mutant of one of the strains Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; using the deposited strain as starting material and applying mutagenesis, wherein the obtained mutant retains or enhances the probiotic and/or antimycotic and/or antimicrobial properties and/or the capacity to inhibit mycosis.

In an embodiment of the present invention the composition may be free from, or substantially free from, viable microorganisms. The composition can comprise cell material including dead cells in form of a lysate, a ferment or a ferment-lysate. In a further embodiment of the present invention the composition may comprise the supernatant from fermentation and the cell material having a further functional effect.

In an embodiment of the present invention the lactic acid bacteria according to the present invention may be capable of inhibiting growth of fungi.

The microorganisms according to the present invention may preferably be in isolated form, where the term “isolated” means in particular that the lactic acid bacteria are derived from their culture medium including their natural medium, for example isolated from nature and/or isolated from other species.

The term “inhibition” or “inhibit” as used herein, means the killing of a microorganism, such as an undesired microorganism, or the control of the growth of said microorganism. Including inhibition of biofilm formation which can be inhibition of the initial attachment of a microorganism or inhibition of the further formation of a biofilm by growth inhibition of the microorganism.

In an embodiment of the present invention the composition and/or the lactic acid bacteria according to the present invention may inhibit growth of fungi.

In an embodiment of the present invention the composition and/or the lactic acid bacteria according to the present invention may inhibit mycoses.

In an embodiment of the present invention the composition and/or the lactic acid bacteria according to the present invention may inhibit dermatophytoses.

An embodiment of the present invention relates to an isolated bacterial strain selected from Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; or a combination hereof wherein said strains presents antimicrobial activity.

The antifungal activity may be normalizing a dysfunctional microbiota and/or inhibition of fungi overgrowth and/or inhibition of mycoses. In particular, the isolated bacterial strain selected from Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; or a combination hereof may inhibit growth of one or more fungi. More specifically, the isolated strains may inhibit growth of pathogenic fungi. Even more specifically, the isolates strains may inhibit mycoses. Even more specifically the isolates may inhibit dermatophytosis.

In a preferred embodiment of the present invention the antifungal activity may be determined as an inhibition of fungal spore germination. More specifically, the antifungal activity may be determined as inhibition of hyphae formation (fungal hyphae formation).

In the present context the inhibition of hyphae formation may be an at least 10% hyphae inhibition, such as at least 25% hyphae inhibition, such as an at least 50% hyphae inhibition, e.g. an at least 75% hyphae inhibition, such as an at least 85% hyphae inhibition, e.g. an at least 90% hyphae inhibition, such as an at least 95% hyphae inhibition, e.g. an at least 98% hyphae inhibition.

Anti-fungal activity can be determined as growth inhibition wherein fungal growth inhibition is determined as inhibition zones when the anti-fungal probiotic strain may be spotted on a lawn of fungi grown on agar plates.

Anti-fungal activity can be determined as growth inhibition wherein fungal growth inhibition may be determined as inhibition zones when the anti-fungal probiotic strain is added in a well stamped into agar with a lawn of fungi grown on the agar plates.

The inventors of the present invention identified and isolated the specific strains of Lactiplantibacillus plantarum LB990R; Lactiplantibacillus plantarum LB681R; Lacticaseibacillus paracasei LB857R; Lactiplantibacillus plantarum LB760R; Lacticaseibacillus paracasei subsp. paracasei LB555R9; and surprisingly identified one or more specific activities provided by the specific strains, like inhibition of fungal growth, and/or for the treatment of dysfunctional microbiota, and/or prevention of mycoses, and/or treatment of mycoses.

The inventors of the present invention identified and isolated the specific strains of Lactiplantibacillus plantarum LB990R; Lactiplantibacillus plantarum LB681R; Lacticaseibacillus paracasei LB857R; Lactiplantibacillus plantarum LB760R; Lacticaseibacillus paracasei subsp. paracasei LB555R; and surprisingly identified one or more specific activities provided by the specific strains, like inhibition of dermatophytoses, and/or for the treatment of dermatophytosis, and/or prevention of dermatophytosis.

In an embodiment of the present invention, the composition according to the present invention may be used for inhibiting fungal growth on a surface. Preferably, the surface may be a surface of a biological material, and/or a surface of a human or animal. In an embodiment of the present invention, the composition according to the present invention may be used for inhibiting fungal growth on a surface. Preferably, the surface may be a surface of a biological material, and/or a surface of a plant, food, feed, seed or corp.

A preferred embodiment of the present invention relates to a method for reducing and/or inhibiting spore germination of a fungus and/or hyphae formation of a fungus on a surface, the method comprises application of the composition according to the present invention to the surface.

The surface may be a surface infected with a fungus or the composition of the present invention may be applied to a surface the prevent the surface to be infected with fungus.

Preferably, the surface may be the skin of a human or an animal, and/or the surface may be a surface of crops, seeds, food or feed.

The surface infected with fungus or prevention of the surface to be infected with fungus may preferably be a surface infected with one or more dermatophyte or prevent the surface to be infected with one or more dermatophyte.

In an embodiment of the present invention, the composition according to the present invention may be used for inhibiting fungal growth on a surface. Preferably, the surface may be a surface in a hospital, and/or a surface of a medical device.

In yet an embodiment of the present invention, the composition according to the present invention may be used for inhibiting fungal formation in or on the human or animal body.

In an embodiment of the present invention the composition (or the lactic acid bacteria) according to the present invention may be formulated or used in a medicament, a pharmaceutical, a food product, a feed product, a disinfectant, a cosmetic or in a personal care product.

The disinfectant according to the present invention may be used for treating a surface to avoid fungal growth on said surface. The surface may be a medical or a non-medical surface. The medical surface may be a surface in a hospital or the surface of a medical device. The medical device may include a medical device to be implanted into, or used inside a human or an animal, a rectal insert, vaginal insert, dental insert, or a medical device mainly used outside the human or animal body.

The composition according to the present invention, and/or the one or more of the isolated bacterial strains of the present invention may be used in the treatment of one or more of the infections which has arisen from: surgical wounds, decubitus ulcers, infections from catheters, stents, cardiocirculatory devices, prostheses, prosthetic insertions, otologic, orthopaedic and dental prostheses, screws and nails, oral cavity infections and infections of the oral and vaginal mucosa, local infections, otitis, rhinosinusitis, pharyngitis, laryngitis and pneumonia.

In an embodiment of the present invention the composition according to the present invention and/or the one or more of the isolated bacterial strains of the present invention may be effective against antibiotic resistant fungi. In particular, effective against an antibiotic resistant infection.

The use of the composition (or the lactic acid bacteria) according to the present invention as a disinfectant may reduce the risk of fungal infection, in particular reduce the risk of infection by pathogenic fungi, even more particularly, reduce the risk of infection by fungi.

The use of the composition (or the lactic acid bacteria) according to the present invention as a disinfectant may reduce the risk of cutaneous fungal infection, in particular reduce the risk of infection by dermatophytes, even more particularly, reduce the risk of infection by the genera Arthoderma, Lophophyton, Nannizzia, Epidermophyton, Microsporum, debrayomyces, Kluyveromyces and Trichophyton.

An embodiment of the present invention relates to one or more bacterial strains selected from one or more Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; or a combination hereof for use as a medicament.

In an embodiment of the present invention the composition of the invention consisting essentially of one or more bacterial strains selected from Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lacticaseibacillus paracasei LB857R, deposited under the assession number DSM 34493 by Lactobio A/S on Jan. 10, 2023; Lactiplantibacillus plantarum LB760R, deposited under the assession number DSM 34492 by Lactobio A/S on Jan. 10, 2023; Lacticaseibacillus paracasei subsp. paracasei LB555R, deposited under the assession number DSM 34249 by Lactobio A/S on May 5, 2022; or a combination hereof

In the context of the present invention, the term “consisting essentially of”, relates to a limitation of the scope of a claim to the specified features or steps and those features or steps, not mentioned and that do not materially affect the basic and novel characteristic(s) of the claimed invention.

A preferred embodiment of the present invention relates to a composition comprising one or more bacterial strains selected from one or more lactic acid bacteria for use in the prevention and/or treatment of mycoses in a human or in an animal.

Preferably, the prevention and/or treatment of mycosis in a human or in an animal may be prevention and/or treatment of mycoses of or in the skin; prevention and/or treatment of mycoses in the mouth; and/or prevention and/or treatment of mycoses the gut, and/or prevention and/or treatment of mycoses the intestines.

The present invention may relate to a composition consisting essentially of one or more bacterial strains selected from one or more of the following strains Lactiplantibacillus plantarum LB990R, deposited under the assession number DSM 34494; Lactiplantibacillus plantarum LB681R, deposited under the assession number DSM 34250; Lacticaseibacillus paracasei LB857R, deposited under the assession number DSM 34493; Lactiplantibacillus plantarum LB760R, deposited under the assession number DSM 34492; Lacticaseibacillus paracasei subsp. paracasei LB555R deposited under the assession number DSM 34249 or a combination hereof for use in the prevention and/or treatment of mycoses in a human or in an animal.

The present Invention may relate to a composition consisting essentially of one or more bacterial strains selected from one or more of the following strains Lactiplantibacillus plantarum LB990R, deposited under the assession number DSM 34494; Lactiplantibacillus plantarum LB681R, deposited under the assession number DSM 34250; Lacticaseibacillus paracasei LB857R, deposited under the assession number DSM 34493; Lactiplantibacillus plantarum LB760R, deposited under the assession number DSM 34492; Lacticaseibacillus paracasei subsp. paracasei LB555R deposited under the assession number DSM 34249; or a combination hereof for use in the prevention of fungal growth for preservation of food, food products, animal feed, corp or seeds.

In an embodiment of the present invention the antifungal activity is against at least one of the fungal species causing cutaneous mycoses.

In an embodiment of the present invention the antifungal activity may be against dermatophytes a fungi classified as Arthrodermataceae are a family of fungi containing seven dermatophyte genera; Epidermophyton, Microsporum, Nannizzia, Trichophyton, Paraphyton, Lophophyton and Arthroderma.

In an embodiment of the present invention the antifungal activity may be against at least one of the following fungal genera: Arthroderma, Lophophyton, Nannizzia, Epidermophyton, Microsporum, Debrayomyces, Kluyveromyces, Candida, Pichia and Trichophyton.

In an embodiment of the present invention the antifungal activity may be against species of the genus Trichophyton.

In the context of the present invention the term “virulence” relates to the quality or property of the pathogenic fungi of being poisonous, venomous or injurious to life or wellbeing of a human or an animal.

The composition according to the present invention may preferably consist essentially of one or more bacteria strain selected from a Lactocaseibacillus strain.

The one or more bacteria strain selected from Lactocaseibacillus may preferably comprise Lacticaseibacillus paracasei subsp. paracasei LB555R, deposited under the assession number DSM 34249 and/or Lacticaseibacillus paracasei LB857R, deposited under the assession number DSM 34493.

The composition according to the present invention may consist essentially of one or more bacteria strain selected from a Lactiplantibacillus strain.

The one or more bacteria strain selected from Lactiplantibacillus may preferably comprise Lactiplantibacillus plantarum LB681R, deposited under the deposit assession number DSM 34250; and/or Lactiplantibacillus plantarum LB760R, deposited under the assession number DSM 34492; and/or Lactiplantibacillus plantarum LB990R, deposited under the assession number DSM 34494.

The composition according to the present invention may consist essentially of one or more bacteria strain selected from a Lacticaseibacillus strain and a Lactiplantibacillus strain.

The composition may be formulated for oral use, topical use, intestinal use or genital use.

Preferably, the composition according to the present invention is formulated for oral or genital use. Even more preferably, the composition according to the present invention is formulated for genital use.

In an embodiment of the present invention the genital use may be female genital use, such as vaginal use, and/or male genital use. Preferably, the genital use may be vaginal use.

During topical use of the composition of the present invention the composition may be used on the skin, in folds of the skin, on nails, on fingernails, in the eye, in the ear, in the hair, on the feet, on the hands, and in the intestine, gut or mucous membranes, such as the mouth of a human or animal.

In an embodiment of the present invention the composition for topical use may be as described in WO2020127637 or WO2020249734.

In an embodiment of the present invention the composition may be formulated as a suspension, spray, gel, cream, lotion, powder, capsule, oil, solution for lavages, ovules, a vaginal insert, a suppository, lozenge, tablets, microencapsulated product or in form of a food supplement or a food product.

The suspension, spray, gel, cream, lotion, powder, capsule, oil, solution for lavages, ovules, vaginal insert, suppository, lozenge, tablets, microencapsulated product, food supplement or food product, may comprise in the range of 0.1-15% (w/w) of the composition according to the present invention, such as in the range of 0,25-12% (w/w), e.g. in the range of 0.50-10% (w/w), such as in the range of 0,75-8% (w/w), e.g. in the range of 1-6% (w/w), such as in the range of 2-5% (w/w), e.g. in the range of 3-4% (w/w).

The suspension, spray, gel, cream, lotion, powder, capsule, oil, solution for lavages, ovules, vaginal insert, suppository, lozenge, tablets, microencapsulated product, food supplement or food product, may comprise in the range of 1×10⁴ CFU/g to 5×10¹¹ CFU/g of the composition according to the present invention, such as in the range of 1×10⁵ CFU/g to 1×10¹¹ CFU/g, e.g. in the range of 5×10⁴ CFU/g to 5×10¹⁰ CFU/g, such as in the range of 1×10⁵ CFU/g to 1×10¹⁰ CFU/g, e.g. in the range of 5×10⁵ CFU/g to 5×10⁹ CFU/g.

In an embodiment of the present invention the composition may be formulated as a cream, in particular a foot cream and foot cream may comprise in the range of 0.1-15% (w/w) of the composition according to the present invention, such as in the range of 0,5-10% (w/w), e.g. in the range of 1-8% (w/w), such as in the range of 3-6% (w/w), e.g. about 5% (w/w). Preferably, the composition may be provided in the form of a lysate (or ferment-lysate).

In an embodiment of the present invention the composition may be formulated as a cream, in particular a foot cream and foot cream may comprise in the range of 1×10⁴ CFU/g to 5×10¹¹ CFU/g of the composition according to the present invention, such as in the range of 1×10⁵ CFU/g to 1×10¹¹ CFU/g, e.g. in the range of 5×10⁴ CFU/g to 5×10¹⁰ CFU/g, such as in the range of 1×10⁵ CFU/g to 1×10¹⁰ CFU/g, e.g. in the range of 5×10⁵ CFU/g to 5×10⁹ CFU/g. Preferably, the composition may be provided in the form of a lysate (or ferment-lysate).

In a further embodiment of the present invention the composition may be formulated as an oil, in particular a foot oil and/or a nail oil, and the oil may comprise in the range of 0.1-15% (w/w) of the composition according to the present invention, such as in the range of 0,25-10% (w/w), e.g. in the range of 0.5-8% (w/w), such as in the range of 0.75-6% (w/w), e.g. in the range of 1-4% (w/w), such as in the range of 1.5-2.5% (w/w), e.g. about 2% (w/w). Preferably, the composition may be provided in the form of a viable freeze-dried cells.

In a further embodiment of the present invention the composition may be formulated as an oil, in particular a foot oil and/or a nail oil, and the oil may comprise in the range of 1×10⁴ CFU/g to 5×10¹¹ CFU/g of the composition according to the present invention, such as in the range of 1×10⁵ CFU/g to 1×10¹¹ CFU/g, e.g. in the range of 5×10⁴ CFU/g to 5×10¹⁰ CFU/g, such as in the range of 1×10⁵ CFU/g to 1×10¹⁰ CFU/g, e.g. in the range of 5×10⁵ CFU/g to 5×10⁹ CFU/g. Preferably, the composition may be provided in the form of a viable freeze-dried cells.

In yet an embodiment of the present invention the composition may be formulated as a powder, in particular a foot powder and/or a skin or scalp powder, and the powder may comprise in the range of 0.1-15% (w/w) of the composition according to the present invention, such as in the range of 0,2-10% (w/w), e.g. in the range of 0.3-8% (w/w), such as in the range of 0.5-6% (w/w), e.g. in the range of 0.6-4% (w/w), such as in the range of 0.75-2.5% (w/w), e.g. about 1% (w/w) or about 2% (w/w). Preferably, the composition may be provided in the form of a viable freeze-dried cells.

In yet an embodiment of the present invention the composition may be formulated as a powder, in particular a foot powder and/or a skin or scalp powder, and the powder may comprise in the range of 1×10⁴ CFU/g to 5×10¹¹ CFU/g of the composition according to the present invention, such as in the range of 1×10⁵ CFU/g to 1×10¹¹ CFU/g, e.g. in the range of 5×10⁴ CFU/g to 5×10¹⁰ CFU/g, such as in the range of 1×10⁵ CFU/g to 1×10¹⁰ CFU/g, e.g. in the range of 5×10⁵ CFU/g to 5×10⁹ CFU/g. Preferably, the composition may be provided in the form of a viable freeze-dried cells.

A preferred embodiment of the present invention relates to a food or feed ingredient, or a food or feed product, comprising the composition according to the present invention.

A preferred embodiment of the present invention relates to a personal hygiene product comprising the composition according to the present invention.

A preferred embodiment of the present invention relates to a cosmetic product comprising the composition according to the present invention.

The composition according to the present invention may be supplemented with further components to improve the effect of the composition, or to stabilize the composition, or both.

In an embodiment of the present invention the composition further comprises an antimycotic drug. This additional antimycotic drug may further improve the activity of the composition in preventing or inhibiting biofilm formation.

The composition according to the present invention may be formulated as a composition for topical use.

The composition according to the present invention may be formulated into medical device. Thus, the composition according to the present invention comprising one or more lactic acid bacteria according to the invention, may be used as a medical device.

The medical device according to the present invention may be formulated into a suspension, spray, gel, cream, lotion, powder, capsule, ointment, oil, solution for lavages, ovules, a vaginal insert, a suppository, capsules, lozenge, tablets, microencapsulated product.

In a further embodiment of the present invention the composition further comprises one or more prebiotic. The prebiotic may improve viability of the added lactic acid bacteria and/or the naturally present microbiota.

In an embodiment of the present invention the composition comprising the lactic acid bacteria may further comprise a prebiotic.

Prebiotics are non-digestible food components that increase the growth of specific microorganisms. “Synbiotics” are compositions comprising at least one probiotic and at least one prebiotic. Such compositions are understood to encourage the growth of beneficial bacteria (e.g. the probiotic). Thus, powerful synbiotics are based on a combination of specific strains of probiotic bacteria with carefully selected prebiotics. They can lead to an important health benefit to a human or in an animal.

An embodiment of the present invention the composition according to the present invention may comprise a symbiotic composition.

According to another embodiment of the present invention there may be provided a probiotic composition comprising the probiotic microorganism and at least one more active ingredient.

Prebiotics refer to chemical products that induce the growth and/or activity of commensal microorganisms (e.g., bacteria and fungi) that contribute to the well-being of their host. Prebiotics are nondigestible carbohydrates that are undigested or partly un-digested by the host and stimulate the growth and/or activity of advantageous bacteria that colonize the host.

Some oligosaccharides that may be used as prebiotics are fructo-oligosaccharides (FOS), xylooligosaccharides (XOS), polydextrose, pectins, galacto-oligo saccharides (GOS) or human milk oligosaccharides (HMO). Moreover, disaccharides like lactulose or lactose some monosaccharides such as tagatose can also be used as prebiotics.

The other active ingredient (or other ingredients) may not be limited in any way.

Prebiotics may be compounds which can be metabolized by probiotics. Preferably prebiotics are non-digestible or poorly digestible by a mammal. Prebiotics are well known in the art and when used in the present invention there is no particular limitation of the prebiotic as such.

In an embodiment of the present invention, at least one prebiotic may be added to the composition. Preferably, the at least one prebiotic may be selected from the following compounds and compositions: non-digestible carbohydrates, beta-glucans, mannan-oligosaccharides, inulin, oligofructose, human milk oligosaccharides (HMO), galactooligosaccharides (GOS), lactulose, lactosucrose, galactotriose, fructo-oligosaccaride (FOS), cellobiose, cellodextrins, cylodextrins, maltitol, lactitol, glycosilsucrose. Optionally, mannan-oligosaccharides and/or inulin may be preferred.

HMOs may include lacto-N-tetraose, lacto-N-fucopentaose, lacto-N-triose, 3′-sialyllactose, lacto-N-neofucopentaose, sialic acid, L-fucose, 2-fucosyllactose, 6′-sialyllactose, lacto-N-neotetraose and 3-fucosyllactose.

D- and L-fucose are considered to strengthen natural defence of skin, stimulate epidermis immune defence and/or prevent and/or treat cutaneous autoimmune disease. Thus, in an embodiment of the present invention the composition may comprises D- and/or L-fucose.

In yet an embodiment of the present invention the composition comprises L-fucose in a concentration in the range of 1 mM to 1000 mM, such as in the range of 10 mM to 500 mM, e.g. in the range of 25 mM to 250 mM.

The composition according to the present invention may further comprise at least one active ingredient.

In an embodiment of the present invention the composition may comprise at least one further probiotic microorganism selected from the group consisting of bacteria, yeasts or molds.

The term “proliferation” as used in the present invention may relate to the ability of a cell to grow and divide into two or more identical cells.

The term “viable” or “live” as used herein relates to a microorganism which is not dead and are able to have an active metabolism.

The term “microbiota” as used herein relates to communities of commensal, symbiotic and pathogenic microorganisms found in and on all multicellular organisms. Microbiota include bacteria, archaea, protists, fungi, yeast, viruses and phages.

The term “microbiota dysfunction” as used herein relates to a state in which the microbiota functions incorrectly or is obstructed from functioning at all. Unless otherwise specified, a microbiota dysfunction is in view of the present invention including the overgrowth or increase in growth of a pathogenic microorganism resulting in a dysfunctional microbiota. One example of a dysfunctional microbiota is an increase in Malassezia furfur resulting in dandruff.

The term “Probiotic microorganism” as used herein relates to live microorganisms that are intended to have health benefits when consumed or applied to the host. Examples of suitable probiotic microorganisms include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Cutibacterium, Lactiplantibacillus and Lactobacillus.

The most commonly used probiotics are strains of the lactic acid bacteria (LAB). The term “lactic acid bacteria” includes species from the families Lactobacilliae, Aerococcaceae, Bifidobacteriaceae, Carnobacteriaceae, Enterococcaceae, Leuconostocaceae and Streptococcaceae. These are considered non-pathogenic and are used as probiotic bacteria in general to improve gastrointestinal flora and in the treatment of gastrointestinal symptoms.

The lactic acid bacteria is preferably selected among the genera Lactobacillus, Lactiplantbacillus, Holzapfelia, Amylolactobacillus, Bombilactobacillus, Companilactobacillus, Lapidilactobacillus, Agrilactobacillus, Schleiferilactobacillus, Loigolactobacillus, Lacticaseibacillus, Latilactobacillus, Dellaglioa, Liquorilactobacillus, Ligilactobacillus, Furfurilactobacillus, Paucilactobacillus, Limosilactobacillus, Fructilactobacillus, Acetilactobacillus, Apilactobacillus, Levilactobacillus, Secundilactobacillus, Lentilactobacillus, Leuconostoc, Bifidobacterium, Pediococcus, Lactococcus, Streptococcus, Aerococcus, Carnobacterium, Enterococcus, Oenococcus, Sporolactobacillus, Tetragenococcus, Vagococcus, and Weissella.

The preferred lactic acid bacteria are in particular bacteria. The bacteria is preferably selected from the group comprising Lactococcus lactis, Lacticaseibacillus rhamnosus, Lactiplantibacillus plantarum, Lactobacillus helveticus, Lactobacillus jensenii, Lactobacillus acidophilus, Lactobacillus bulgaricus, Lactobacillus amylovorus, Lactobacillus amylolyticus, Lactobacillus alimentarius, Lactobacillus aviaries, Lactobacillus delbrueckii, Lactobacillus diolivorans, Lactobacillus farciminis, Lactobacillus gallinarum, Lacticaseibacillus casei, Lactobacillus crispatus, Lactobacillus gasseri, Lactobacillus johnsonii, Lactobacillus hilgardii, Lactobacillus kefiranofaciens, Lactobacillus kefiri, Lactobacillus mucosae, Lactobacillus panis, Lactiplantibacillus paraplantarum, Lactobacillus pontis, Latilactobacillus sakei, Lactobacillus saliverius, Lactobacillus sanfraciscensis, Lacticaseibacillus paracasei, Lactobacillus pentosus, Lactobacillus cellobiosus, Lactobacillus collinoides, Lactobacillus coryniformis, Lactobacillus curvatus, Levilactobacillus brevis, Lactobacillus buchneri, Lactobacillus fructivorans, Lactobacillus hilgardii, Lactobacillus fermentum, Lactobacillus reuteri, Lactobacillus ingluviei, Weissella viridescens, Bifidobacterium bifidum, Bifidobacterium adolescentis, Bifidobacterium breve, Bifidobacterium longum, Bifidobacterium animalis, Carnobacterium divergens, Corynebacterium glutamicum, Leuconostoc citreum, Leuconostoc lactis, Leuconostoc mesenteroides, Leuconostoc pseudomesenteroides, Oenococcus oeni, Pasteuria nishizawae, Pediococcus acidilactici, Pediococcus dextrinicus, Pediococcus parvulus, Pediococcus pentosaceus, Probionibacterium freudenreichii, Probionibacterium acidipropoinici, Enterococcus faecium, Enterococcus faecalis, Streptococcus thermophilus, Bacillus amyloliquefaciens, Bacillus atrophaeus, Bacillus clausii, Bacillus coagulans, Bacillus flexus, Bacillus fusiformis, Bacillus lentus, Bacillus licheniformis, Bacillus mega-terium, Bacillus mojavensis, Bacillus pumilus, Bacillus smithii, Bacillus subtilis, Bacillus vallismortis, Geobacillus stearother-mophilus or mutants thereof.

In one preferred embodiment of the invention the composition comprises at least one further strain selected from the group consisting of Lactiplantibacillus plantarum LB356R (DSM 33094), Lactiplantibacillus plantarum LB244R (DSM 32996), Weissella viridescens LB10G (DSM 32906), Lacticaseibacillus paracasei LB113R (DSM 32907), Lacticaseibacillus paracasei LB116R (DSM 32908), Levilactobacillus brevis LB152G (DSM 32995), Lacticaseibacillus paracasei LB28R (DSM 32994), Enterococcus faecium LB276R (DSM 32997), Leuconostoc mesenteriodes LB349R (DSM 33093), Lactiplantibacillus plantarum LB316R (DSM 33091), Lactiplantibacillus plantarum LB312R (DSM 33098), Pediococcus pentosaceus LB606R (DSM 33730), Lactiplantibacillus plantarum LB679R (DSM 33731), Lactobacillus crispatus LB714R (DSM 33732), Lactobacillus gasseri LB905R (DSM 34094), Lactobacillus crispatus LB912R (DSM 34095), Lactobacillus jensenii LB918R (DSM 34096), Lactobacillus crispatus LB919R (DSM 34097); and/or any combinations hereof or mutant strains thereof and/or the cell lysate and/or the soluble metabolite of anyone of these probiotic strains.

The number of microorganisms is measured as Colony Forming Units (CFU) per ml or per gram.

The microorganisms according to the present invention may preferably be in isolated or purified form, where the term “isolated” means in particular that the lactic acid bacteria are derived from their culture medium including their natural medium, for example. The terms “isolated” and “purified” may not restricted to absolute purity.

In an embodiment of the present invention the probiotic strain may be used as a live isolated microorganism in a stabilized form. Suitable methods for stabilization are known to those skilled in the art and includes freeze drying or lyophilization involving different cryoprotectants.

In a further embodiment of the present invention the strain may be used as a live isolated strain.

The term “postbiotic” refers to compounds, metabolites or cell materials secreted or released from a probiotic microorganism providing a health benefit when applied to the host. A postbiotic composition is characterized by having a health benefit without the presence of the viable microorganism.

The term “Postbiotic fraction of a probiotic microorganism” as used herein disclose a fermented composition of the probiotic microorganism substantially free from viable microorganisms. The composition can comprise cell material including dead cells.

In an embodiment of the present invention the probiotic strain may be used as a live isolated microorganism in a stabilized form. Suitable methods for stabilization are known to those skilled in the art and includes freeze drying, spray drying or lyophilization involving different cryoprotectants.

The invention thus further provides a method for controlling, curatively or preventively, the phytopathogenic fungi of crops, characterized in that an effective (agronomically effective) and nonphytotoxic quantity of an antifungal composition according to the invention is applied to the seeds, soil where plants grow or are capable of growing, to the leaves and/or the fruit of plants or to the seeds of plants.

The compositions according to the invention are advantageous for controlling the fungal diseases of numerous crops, such as for example cereals, vegetables, solanaceous plants, market garden crops, grapevine, fruit in general, and in particular mildews, Septoria diseases, Pythium sp. of these crops.

The compositions of the invention generally inhibit fungal growth of a number of fungi that are particularly harmful in crops, in particular for grapevine, more particularly on vine downy mildew.

These compositions cover not only compositions which are ready to be applied to the crop or seed to be treated by means of a suitable device, such as a spraying device, but also commercial concentrated compositions which need to be diluted or mixed with a carrier before they are applied onto the crop.

The present invention provides a biological method for controlling a large variety of phytopathogenic diseases of crops.

The present invention thus provides a method for controlling, curatively or preventively, the phytopathogenic diseases of crops, which comprises treatment of said crop (for example by application or by administration) with an effective and non-phytotoxic amount of a composition of the invention. The expression “treatment of the crop” means an application or administration of a antifungal composition as described above onto, for example, the aerial parts of the crops or onto the soil in which they are growing and which are infested or liable to become infested with a phytopathogenic disease, such as mildew or

Septoria leaf blotch. The expression “treatment of the crop” also means treatment of the reproduction products of the crop, such as the seeds or the tubers, for example.

The compositions described below are used in general for application onto growing vegetation, or onto areas in which crops are grown, or for the coating of or film coating of seeds.

The expression “are applied to the vegetation to be treated” is understood to mean, for the purposes of the present text, that the antifungal composition of the invention may be applied by means of various treatment processes such as: spraying a liquid comprising a composition onto the aerial parts of said vegetation, dusting, incorporation of granules or powders into the soil, watering around said vegetation and, coating of or formation of a film on seeds of said vegetation using a broth or carrier comprising a composition.

In addition to the control of mycoses and crop protection a further embodiment of the invention is inhibition or growth control of fungi causing food spoilage or other concerns in the food processing industry. In a preferred embodiment of the invention the lactic acid bacteria and/or a ferment lysate are able to control the growth of “spoilage” fungi. As used herein, the term “spoilage fungi” refers to any food or feed fungi of causing disease or illness in animals or humans and/or causing food spoilage. The term spoilage fungi will be understood to include fungi with the ability to grow and/or spoil edible substances and thereby can cause spoilage, disease or illness after consumption by mammals, as well as fungi that produce toxins that cause disease or illness. The proliferation of spoilage fungi on food or feed products can cause severe illness and may be deadly, as demonstrated by the number of human fatalities caused by food poisoning. The term “undesired fungi” as used herein, refers to both spoilage and pathogenic fungi.

The term “edible substance” or “edible product” refers to a substance or product safe to oral consumption by humans or animals, which encompass food and feed products as well as ingredients for food or feed products.

The term “food product” as used herein refers to any food that is susceptible to spoilage as a result of microbial growth and proliferation on the surface of the food. Such food products include, but are not limited to meat, dairy products, vegetables, fruits and grains.

As used herein, the term “meat” refers to any fresh meat product, processed meat or meat by-product from an animal of the kingdom Animalia which is consumed by humans or animals, including without limitation meat from bovine, ovine, porcine, poultry, frog, fish and crustaceous seafood. Thus, while one of the primary uses for the present invention relates to meat processed in the slaughtering of mammals in a meat processing facility, it is to be expressly understood that the invention has application in the processing of other edible meat products including fish, poultry and seafood as well as cultured meat. Also included is dairy products including hard cheeses, yogurt and dairy products for which fungal growth is unwanted. Moreover, it is contemplated that the method also will have use in connection with the preservation of non-animal food products, such as fruits, vegetables, bread, bread products, cakes, grains and grain based products, subject to spoilage by microorganisms.

In a preferred embodiment the meat is cultured meat. Besides “cultured meat”, the terms healthy meat, slaughter-free meat, in-vitro meat, vat-grown, lab-grown meat, cell-based meat, clean meat, cultivated meat and synthetic meat have all been used by various outlets to describe such product. Typically cultured meat is grown from steam cells. Some of the first descriptions of cultured meat is in U.S. Pat. No. 6,835,390 B1 with the production of tissue-engineered meat for human consumption, wherein muscle and fat cells would be grown in an integrated fashion to create food products such as beef, poultry and fish.

It will be clear to those skilled in the art that here, as well as in all the statements of range given in the present invention, characterized by such terms as “about” or “approximately,” that the precise numerical range need not be indicated with expressions such as “about” or “approx.” or “approximately,” but instead even minor deviations up or down with regard to the number indicated are still within the scope of the present invention.

The composition according to the present invention may comprise at least one bacterial strain in combination with at least one further probiotic microorganism, wherein the at least one further probiotic microorganism may be selected from but not restricted to a strain selected from the genus Lactobacillus.

### Deposit of Biological Material

The lactic acid bacteria according to the present invention include in particular microorganisms or analogs, fragments, derivatives, fermentates, lysates, ferment-lysates, mutants or combinations obtained from the microorganism, where the microorganism is deposited on 5 of May 2022 with the German Collection for Microorganisms and Cell Lacticaseibacillus paracasei subsp. paracasei LB555R, deposited under the assession number DSM 34249, and Lactiplantibacillus plantarum, LB681R, deposited under the assession number DSM 34250, and Lactiplantibacillus plantarum LB760R, deposited under the assession number DSM 34492, and Lactiplantibacillus plantarum LB990R, deposited under the assession number DSM 34494, and Lacticaseibacillus paracasei LB857R, deposited under the assession number DSM 34493.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

### Strain Screening Identification

For identification and selection of microorganisms according to the invention, a strain collection of new isolated lactic acid bacteria (LAB) was established. Samples from different origins, such as homemade sauerkraut, sourdough, kimchi, fermented food and healthy human donor samples (vaginal, oral, anal, skin, human milk, baby diapers) were collected for isolation of at least 1500 new lactic acid bacteria strains. The samples were collected on Man Rogosa Sharp (MRS, Sigma-Aldrich) broth and agar cultured anaerobically at 37° C. overnight or until colony formation. The new isolates are plated and sub-cultured until isolated colonies were obtained. The isolated colonies are stored in MRS broth with 25% glycerol at −80° C. for future use. Strains were identified using 16S rRNA Sanger sequencing standard methods.

### Example 2

Inhibition of M. furfur DSMZ 6170, M. restricta CBS 7877 and M. globosa CBS 7874

Inhibition was assessed using a spot-on lawn method adapted from Zhang P. et al. (2015) Inter-strain interactions between bacteria isolated from vacuum-packaged refrigerated beef. Appl Environ Microbiol 81: 2753-2761. doi: 10.1128/AEM.03933-14 and Arena, M. P. et al. (2016) Use of Lactobacillus plantarum Strains as a Bio-Control Strategy against Food-Borne Pathogenic Microorganisms. Frontiers in Microbiology 7 (APR): 1-10. doi: 10.3389/fmicb.2016.00464.

Target strain Malassezia furfur DSMZ 6170 was obtained from Leibniz Institute DSMZ. Target strains M. globosa CBS 7874 and M. restricta CBS 7877 were obtained from Westerdijk fungal biodiversity institute CBS. Target strains were grown in modified Leeming Notmann (mLN) (ATCC Medium No. 2737 Leeming & Notman agar Modified) agar and single colonies were inoculated into 10 mL of mLN Broth. A lawn was created using the target strain culture and allowed to dry. Bacterial strains isolated from example 1 were cultured from storage in 10 mL MRS broth. Subsequently, overnight cultures of LAB were spotted onto the plates and allowed to incubate. M. furfur was grown at 30° C. for approximately 10 days, M. restricta was grown at 30° C. for approximately 14 days and M. globosa was grown at 33° C. for approximate 14 days. Inhibition zones were measured from the edge of the LAB colony to the beginning of visible Malassezia growth (clearing zones) in millimeters. All plates were done in technical duplicates and repeated on different occasions.

LAB were screened against M. furfur DSMZ 6170, of which 84 showed moderate inhibition and 18 showed high inhibition. 2 strains were selected from the 18 strains and subsequently tested using the spot-on lawn method against M. restricta CBS 7877 and M. globosa CBS 7874. Both strains were capable of inhibiting M. restricta CBS 7877 and M. globosa CBS7874.

TABLE 1
Spot assay with Malassezia spp. As target, inhibition/clearing
zones measured in millimeters as an average of duplicates
between the periphery of the bacterial colony and visible
growth of Malassezia spp.

| | Inhibition zone - | Inhibition zone - | Inhibition zone - |
|---|---|---|---|
| | M. furfur DSMZ | M. restricta CBS | M. globosa CBS |
| Strain | 6170 | 7877 | 7874 |
| L. paracasei | 3.0 | 20 | 21.5 |
| LB555R | | | |
| L. plantarum | 4.5 | 30 | 17.5 |
| LB681R | | | |

### Example 3

Trichophyton spp. spot on agar assay were performed and anti-fungal activity against Trichophyton spp. were determined as described in example 2.

Trichophyton rubrum CBS 189.69 a nail isolate was obtained from the Westerdijk Fungal Biodiversity Institute part of the Royal Netherlands Academy of Arts and Sciences.

Trichophyton rubrum CBS 392.58 (Neotype of Epidermophyton rubrum Castell) a skin (foot) isolate was obtained from the Westerdijk Fungal Biodiversity Institute part of the Royal Netherlands Academy of Arts and Sciences.

Trichophyton spp. was grown on Sabouraud maltose agar at 24° C. for 2 to 3 weeks.

TABLE 2
Spot assay with Trichophyton spp. As target, inhibition/clearing
zones measured in millimeters as an average of duplicates
between the periphery of the bacterial colony and visible
growth of Trichophyton spp

| | Inhibition zone - | Inhibition zone - |
|---|---|---|
| | T. rubrum CBS | T. rubrum CBS |
| Strain | 189.69 | 392.58 |
| L. paracasei | 28 | 23 |
| LB555R | | |
| L. plantarum | 35 | 25 |
| LB681R | | |

### Example 4

### Inhibition by Cell-Free Supernatant Solution

Growth of Malassezia spp. (2 strains as described in example 2) with and without cell free supernatant (CFS) of LAB from example 2 was monitored using the oCelloScope (BioSense Solutions ApS, Denmark).

Between 5 to 10 mL of LAB cell culture was centrifuged (2,700× g, 10 min) and sterile filtered using a 0.2 μm syringe filter. Samples were taken from the LAB-cultures after 24-72 hours.

From 10 mL of Malassezia spp. Cultures a 1:2 dilution was prepared. From each of these dilutions, 100 μL was mixed with 100 μL CFS or dilutions thereof using of 100, 75, 50, 25, 10 and 0% CFS in a 96 wells microtiter plate. The volume was adjusted using MRS broth. As a positive control for inhibition, Malassezia spp. Were treated with fluconazole (64 μg/mL). All challenge assays were run in technical triplicates and repeated on separate occasions. The plate was incubated in the oCelloScope at 37° C. Raw images were taken every 2 and a half hours for up to 48 hours and growth curves were plotted in excel, calculated as an average of the triplicates.

Post-experiment, plates were visually inspected, and raw images were visually examined. Images were analysed using UniExplorer PC software.

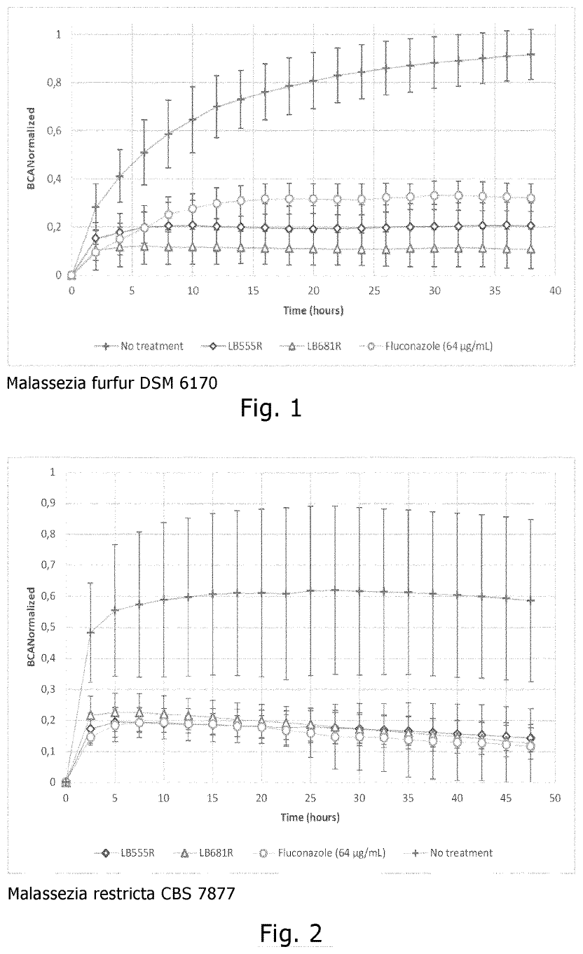

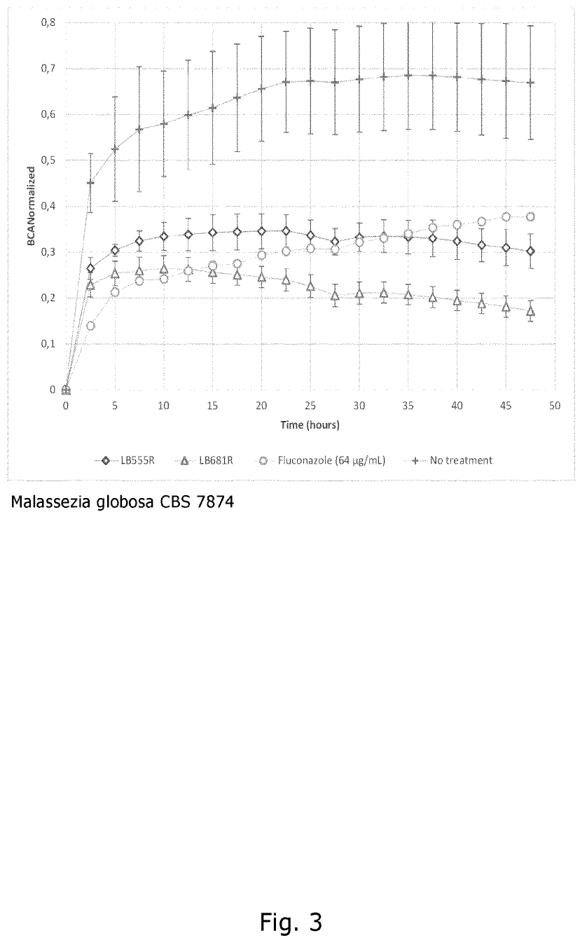

The results are shown in FIGS. 1-3 and demonstrated that the cell free supernatants obtained from the two isolated strains have a strong and significant inhibition of Malassezia furfur DSM 6170 (FIG. 1), M. restricta CBS 7877 (FIG. 2) and M. globosa CBS7874 (FIG. 3) using a composition comprising: (i) Lacticaseibacillus paracasei subsp. paracasei LB555R, deposited under the assession number DSM 34249; (ii) Lactiplantibacillus plantarum, LB681R, deposited under the assession number DSM 34250; compared to a composition comprising the anti-fungal compound Fluconazole (in a concentration of 64 μg/ml); and compared to an un-treated fungal solution.

The two 2 selected strains were identified in the screening as lactic acid bacteria able to growth inhibit the fungal growth of at least 2 pathogenic fungi, wherein the growth inhibition was significantly better than what was observed for the known antifungal antibiotic Fluconazole.

### Example 5

### Analysis of Short Chain Fatty Acid Production (SCFA)

Sample analysis was carried out as follows. Cell free supernatant from the strains grown in MRS medium 24 hours at 37° C. were acidified using hydrochloride acid, and deuterium labelled internal standards where added. Analysis was performed using a high polarity column (Zebron™ ZB-FFAP, GC Cap. Column 30 m×0.25 mm×0.25 μm) installed in a GC (7890B, Agilent) coupled with a quadropole detector (5977B, Agilent). The system was controlled by ChemStation (Agilent). Raw data was converted to netCDF format using Chemstation (Agilent), before the data was imported and processed in Matlab R2014b (Mathworks, Inc.) using the PARADISe software described by Johnsen et. al (DOI: 10.1016/j.chroma.2017.04.052). The SCFA method is a GC-MS method specially targeted to short-chain fatty acids using a high polarity column and standards. Concentration of short chain fatty acids (acetic acid) was determined in the supernatant from each strain.

| | LB555R | LB681R | LB857R | LB990R | LB760R |
|---|---|---|---|---|---|
| Acetic acid | 36.5 mM | 40.76 mM | 37.3 mM | 41.1 mM | 40.7 |

### Example 6

### Well Diffusion Assay

Overnight cultures of LAB were prepared. LAB was grown in MRS broth and the fungi tested grown as described in the examples above for spot on lawn. Wells were made in the plates and 50 μL of the LAB cultures were transferred to the wells. After incubation until visible fungal growth, inhibition zones around the wells were examined.

TABLE 3
Spot on lawn and diffusion assay, inhibition
zones are measured in mm.

| | Spot on lawn | | Well diffusion | |
|---|---|---|---|---|
| | (Diameter in mm) | | (Radius mm) | |
| | LB555R | LB681R | LB555R | LB681R |
| T. rubrum CBS | 25 +/− 5 | 30 +/− 8 | 6 +/− 1 | 7 +/− 1 |
| 189.69 | | | | |
| T. rubrum CBS | 12 +/− 4 | 18 +/− 4 | 7 +/− 2 | 6 +/− 2 |
| 392.58 | | | | |
| M. furfur DSMZ | 3 +/− 1 | 4.5 +/− 2 | 5 +/− 1 | 5 +/− 1 |
| 6170 | | | | |

### Example 7

Inhibition of Trichophyton rubrum CBS 189.69 and T. rubrum CBS 392.58

Inhibition was assessed using a spot-on lawn method adapted from Zhang P. et al (2015). Appl Environ Microbiol 81: 2753-2761. Doi: 10.1128/AEM.03933-14 and Arena, M. P. et al. (2016) Frontiers in Microbiology 7 (APR): 1-10. Doi: https://doi.org/10.3389/fmicb.2016.00464.

The Sabouraud maltose agar used in example 3 and 6 was replaced by Mueller Hinton agar allowing for a more standardized even Trichophyton lawn and smaller inhibition zones. Thereby the inhibition zones do not interfere, or overlap, when more than one spot is made on the lawn. Resulting in a more precise comparison of the inhibition zones. Target strains T. rubrum CBS 392.58 and T. rubrum CBS 189.69 were obtained from Westerdijk Fungal Biodiversity institute CBS. Target strains were grown in Malt Extract agar (MEA) or Malt extract broth (MEB). A 50% glycerol solution was used to harvest spores from target strains grown on MEA. This spore-solution was used to create target strain lawn on Mueller-Hinton (MH) agar.

Bacterial strains from example 1 were cultured from storage in 1 mL MRS broth. Subsequently, overnight cultures of LAB were spotted onto the plates and allowed to incubate at 24° C. for approximately 14 days. Inhibition zones were measured from the edge of the LAB colony to the beginning of visible T. rubrum growth (clearing zones) in millimeters. All plates were done in technical duplicates and repeated on different occasions (biological replicates).

226 pre-selected LAB were screened against T. rubrum CBS 392.58 and T. rubrum CBS 189.69 of which approximately 70 showed inhibition of different degrees. 6 strains were identified as having a significant growth inhibitory activity of T. rubrum

TABLE 4
Spot assay with T. rubrum spp. On MH agar. Inhibition/clearing
zones measured in millimeters as an average of 4 biological and
technical duplicates between the periphery of the bacterial colony
and visible growth of two strains of T. rubrum spp.

| | Inhibition zone - | Inhibition zone - |
|---|---|---|
| | T. rubrum CBS | T. rubrum CBS |
| Strain | 392.58 | 189.69 |
| L. plantarum LB760R | 5 | 7.5 |
| (DSM 34492) | | |
| L. paracasei LB857R | 5.25 | 7.5 |
| (DSM 34493) | | |
| L. plantarum LB990R | 6 | 7.5 |
| (DSM 34494) | | |
| L. paracasei LB555R | 2.5 | 2.5 |
| (DSM 34249) | | |
| L. plantarum LB681R | 3.5 | 5 |
| (DSM 34250) | | |

### Example 8

### Inhibition of Fungal Spore Germination by Cell-Free Supernatant Solution

Fungal spore germination of T. rubrum CBS 392.58 with and without CFS of LAB from example 2 were monitored using the oCelloScope (BioSense Solutions ApS (Hirsemarken 1, DK-3520 Farum)).

Between 5 to 10 mL of LAB cell culture was centrifuged (2,700×g, 10 min) and sterile filtered using a 0.2 μm syringe filter. Samples were taken from the LAB-cultures after 72 hours.

50% glycerol spore solution harvested from MEA plates with T. rubrum CBS 392.58 was diluted in MEB (20 μL Spore solution to 105 μL MEB). This was subsequently treated with different concentrations of cell free supernatant (CFS) (75 μL, 50 μL, 25 μL, 10 μL, 5 μL) diluted with MRS broth.

The 226 pre-selected LAB were screened against T. rubrum CBS 392.58 and T. rubrum CBS 189.69 of which approximately 70 showed inhibition of different degrees. 6 strains were identified as having a significant inhibition of fungal spore germination and hyphae formation of the T. rubrum strains.

All challenge assays were run in technical triplicates and repeated on separate occasions. The plate was incubated in the oCelloScope at 24° C. Raw images were taken every 2 hours for up to approximately 100 hours. Post experiments plates were visually inspected, and raw images were visually examined. Inhibition was classified according to the following scale (calculated as an average of triplicates):

| | dose of CFS | | | | | |
|---|---|---|---|---|---|---|
| Strain | 75 μL | 50 μL | 25 μL | 10 μL | 5 μL | 0 μL |
| Acid control | 0 | 2 | 4 | 4 | 4 | 4 |
| LB990R | 0 | 0 | 0 | 0 | 1 | 4 |
| LB760R | 0 | 0 | 0 | 0 | 2 | 4 |
| LB857R | 0 | 0 | 0 | 0 | 2 | 4 |
| LB681R | 0 | 0 | 0 | 0 | 2 | 4 |
| LB555R | 0 | 0 | 0 | 0 | 3 | 4 |

No germination: 0 (0 germinating spores)
Little germination: 1 (1-5 germinating spores out of 30-50 spores)
Germination: 2 (5-10 germinating spores out of 30-50 spores)
High germination: 3 (more than 10 germinating spores out of 30-50 spores)
Full germination: 4 (all spores are germinating >95%)

### Example 9

### Different Formulations

A) A foot cream—Ingredients provided in % (w/w):

Aqua 45%

Cetearyl alcohol 14%

Sodium cetearyl sulfate 10%

Prunus amygdalus dulcis oil 6%

Prunus armeniaca kernel oil 6%

Glycerin 5%

butyrospermum parkii 5%

LB990R ferment lysate 5%

Vitis vinifera 2%

Starch hydroxypropyltrimonimum chloride 1%

Phenoxyethanol 1%

B) A foot oil-Ingredients provided in % (w/w):

Prunus amygdalus dulcis oil 50%

Prunus armeniaca kernel oil 47%

Lactiplantibacillus plantarum LB990R lyophilized (viable freeze-dried concentration of 3×10¹¹ CF/g) 2%

Tocopherol 1%

C) A nail oil-Ingredients provided in % (w/w):

Castor oil 35%

Prunus amygdalus dulcis oil 32%

Prunus armeniaca kernel oil 30%

Lactiplantibacillus plantarum LB681R lyophilized (viable freeze-dried concentration of 4×10¹¹ CFU/g) 1%

Lactiplantibacillus plantarum LB990R lyophilized (viable freeze-dried concentration of 1×10¹¹ CFU/g) 1%

Tocopherol 1%

D) A foot powder-Ingredients provided in % (w/w):

Ingredients:

Talc 50%

Rice starch 45%

Lacticaseibacillus paracasei LB857R lyophilized (viable freeze dried) 2%

E) Skin or scalp powder-Ingredients provided in % (w/w):

Corn starch 95%

Magnesium stearate 4%

Lactiplantibacillus plantarum LB760R lyophilized (viable freeze-dried concentration of 2×10¹⁰ CFU/g) 1%

All formulations were tested for anti-fungal activity against Trichophyton rubrum CBS 189.69 using the spot on lawn test described in example 7.

The formulations were spotted directly on the lawn, 4 spots for each formulation. Anti-fungal activity was determined if no growth of fungi occurs in the spotted area as well as a clearing zone of at least 1 mm was viable around the spotted formulation. The powder formulations were suspended in sterile saline (0.9% W/W) and 10 μL spotted.

The results from the test showed that the anti-fungal activity of the strains were maintained in the formulations, and all formulations resulted in no growth of fungi in the spotted area, the zone of clearance around the spot was determined in mm.

| Formulation | Clearing zone around spotted formulation (mm) |
|---|---|
| A | 2 +/− 1 |
| B | 4 +/− 1 |
| C | 3 +/− 2 |
| D | 6 +/− 2 |
| E | 5 +/− 2 |

### REFERENCES

Dowarah, R., et al. (2018) Selection and characterization of probiotic lactic acid bacteria and its impact on growth, nutrient digestibility, health and antioxidant status in weaned piglets. PLOS ONE, 13(3)
Khare, A., & Tavazoie, S. (2015). Multifactorial Competition and Resistance in a Two-Species Bacterial System. PLOS Genetics, 11(12), 1-21.

1. A composition comprising one or more probiotic bacterial strains, wherein the composition is capable of inhibiting, partly or completely the proliferation of one or more dermatophyte.

2. The composition according to claims 1, wherein the one or more probiotic bacterial strains is one or more isolated probiotic bacterial strains or a mutant hereof.

3. The composition according to claim 1, wherein the composition comprises no plant material and/or no fibre material.

4. The composition according to claim 1, wherein the one or more dermatophyte is selected from Arthroderma, Lophophyton, Nannizzia, Epidermophyton, Microsporum, Debrayomyces, Kluyveromyces, Pichia, Trichophyton, or a combination hereof.

5. The composition according to claim 1, wherein the one or more probiotic bacterial strains may be selected from a Lacticaseibacillus paracasei and/or a Lactiplantibacillus plantarum.

6. The composition according to claim 1, wherein the one or more probiotic bacterial strains is selected from one or more of:
Lactiplantibacillus plantarum LB990R, deposited under the assession number DSM 34494 by Lactobio A/S on Jan 10, 2023;
Lactiplantibacillus plantarum LB681R, deposited under the assession number DSM 34250 by Lactobio A/S on May 5, 2022;
Lacticaseibacillus paracasei LB857R, deposited under the assession number DSM 34493 by Lactobio A/S on Jan. 10, 2023;
Lactiplantibacillus plantarum LB760R, deposited under the assession number DSM 34492 by Lactobio A/S on Jan. 10, 2023;
Lacticaseibacillus paracasei subsp. paracasei LB555R, deposited under the assession number DSM 34249 by Lactobio A/S on May 5, 2022; or
A combination hereof.

7. The composition according to claim 1, wherein the composition comprises one or more viable probiotic bacterial strains, or the composition comprises non-viable probiotic bacterial strains or the composition comprises metabolites, lysate, e.g. a ferment-lysate obtained from the one or more probiotic bacterial strains, or the composition comprises a fraction of the one or more probiotic bacterial strains, or a combination hereof.

8. An antifungal composition comprising one or more probiotic bacterial strains for the inhibition of spore germination of a fungus and/or inhibition of hyphae formation of a fungus.

9. The antifungal composition according to claim 8, wherein the one or more probiotic bacterial strains is selected from one or more Lactiplantibacillus plantarum, one or more Lacticaseibacillus paracasei, or a combination hereof.

10. The antifungal composition according to claim 9, wherein the one or more Lactiplantibacillus plantarum is selected from Lactiplantibacillus plantarum LB990R, deposited under the assession number DSM 34494 by Lactobio A/S on Jan. 10, 2023; Lactiplantibacillus plantarum LB681R, deposited under the assession number DSM 34250 by Lactobio A/S on May 5, 2022; Lactiplantibacillus plantarum LB760R, deposited under the assession number DSM 34492 by Lactobio A/S on Jan. 10, 2023; and/or wherein the one or more Lacticaseibacillus paracasei is selected from Lacticaseibacillus paracasei LB857R, deposited under the assession number DSM 34493 by Lactobio A/S on Jan. 10, 2023; Lacticaseibacillus paracasei subsp. paracasei LB555R, deposited under the assession number DSM 34249 by Lactobio A/S on May 5, 2022; or a combination hereof

11. The antifungal composition according to claim 8, wherein the fungus is a dermatophyte.

12. A method for reducing and/or inhibiting spore germination of a fungus and/or hyphae formation of a fungus on a surface, the method comprises application of the composition according to claim 1 to the surface.

13. An isolated probiotic bacterial strain selected from:
Lactiplantibacillus plantarum LB990R, deposited under the assession number DSM 34494 by Lactobio A/S on Jan. 10, 2023;
Lactiplantibacillus plantarum LB681R, deposited under the assession number DSM 34250 by Lactobio A/S on May 5, 2022;
Lacticaseibacillus paracasei LB857R, deposited under the assession number DSM 34493 by Lactobio A/S on Jan. 10, 2023;
Lactiplantibacillus plantarum LB760R, deposited under the assession number DSM 34492 by Lactobio A/S on Jan. 10, 2023;
Lacticaseibacillus paracasei subsp. paracasei LB555R, deposited under the assession number DSM 34249 by Lactobio A/S on May 5, 2022.

14. The isolated probiotic bacterial strain according to claim 13, wherein the isolated probiotic bacterial strain is capable of inhibiting, partly or completely the proliferation of one or more dermatophyte.

15. A composition comprising Lactiplantibacillus plantarum LB990R, deposited under the assession number DSM 34494; Lactiplantibacillus plantarum LB681R, deposited under the assession number DSM 34250; Lactiplantibacillus plantarum LB760R, deposited under the assession number DSM 34492; Lacticaseibacillus paracasei LB857R, deposited under the assession number DSM 34493; Lacticaseibacillus paracasei subsp. paracasei LB555R deposited under the assession number DSM 34249; or a combination hereof, for use in:
the treatment or prevention of mycoses, preferably the treatment of and/or prevention of mycoses in a human or in an animal;
the treatment of dermatophytosis, preferably the treatment of and/or prevention of dermatophytosis in a human or in an animal; and/or
the prevention, inhibition, and/or treatment of fungal growth on crops, seeds, food or feed.

16. The composition according to claim 1, wherein the composition is formulated as a suspension, spray, gel, cream, lotion, powder, capsule, oil, solution for lavages, ovules, a vaginal insert, a suppository, lozenge, tablets, microencapsulated product.

17. The isolated probiotic bacterial strain according to claim 13, wherein the bacterial strains are viable freeze-dried cells.

18. The isolated probiotic bacterial strain according to claim 13, wherein the bacterial strains are provided as non-viable probiotic bacterial strains, preferably, in the form of metabolites or lysate, e.g. a ferment-lysate.
